(19) United States
(12) Patent Application Publication
(10) Pub. No.: US 2008/0027003 A1
(43) Pub. Date: Jan. 31, 2008

(54) Peptides Useful for Treating Gnrh Associated Diseases

(75) Inventors: Sergey Burov, St. Petersburg (RU); Nava Epstein, Petach-Tikva (IL)

Correspondence Address: Martin D. Moynihan;PRTSI, P.O.Box 16446, Arlington, VA, 22215, US

(73) Assignee: CurePeptide Ltd., Haifa (IL)

(21) Appl. No.: 11/597,520

(22) PCT Filed: May 26, 2005
(86) PCT No.: PCT/IL05/00543

§ 371 Date: Nov. 27, 2006

Related U.S. Application Data
(60) Provisional application No. 60/574,570, filed on May 27, 2004.
(60) Provisional application No. 60/619,962, filed on Oct. 20, 2004.

(51) Int. Cl. A61K 38/10; A61K 38/08; A61P 43/00; C07K 7/06; C07K 7/08

(52) U.S. Cl. 514/13; 514/14; 514/15; 530/326; 530/327

(57) ABSTRACT

A peptide comprising at least one hydrophobic moiety attached to an amino acid sequence capable of binding a GnRH receptor, the amino acid sequence being at least 11 amino acids in length. Also provided are methods of using such peptides for the treatment of GnRH-associated diseases.

### FIELD AND BACKGROUND OF THE INVENTION

The present invention relates to peptides, which can be used to prevent and treat GnRH-associated diseases, such as cancer.

Gonadotropin-releasing hormone (GnRH) or Luteinizing hormone releasing hormone (LHRH) is a hypothalamic decapeptide which controls the reproductive axis [Jiang (2001) J. Med. Chem. 44:453-467]. GnRH mRNA has been found in pituitary and in extrapituitary tissues, such as in the placenta, ovary, myometrium, endometrium and prostate and blood mononuclear cells, indicating an autocrine/paracrine mode of action. The genes for human GnRH types I and II are located in chromosome 8 and chromosome 20, respectively.

The GnRH receptor (GnRH-r) is a member of the rhodopsin-like-G-protein coupled receptor family, which also includes the thyrotropin-releasing hormone receptor [Sealfon (1997) Endocr. Rev. 18:180-205; Probst (1992) DNA Cell Biol. 11:1-20].

GnRH is synthesized and released in a pulsatile manner from hypothalamic neurosecretory cells, reaches pituitary cells by way of a specialized portal system, regulates the synthesis and release of pituitary gonadotropins which, in turn, regulate steroidogenic and gametogenic functions of the gonads. LH stimulates ovulation and corpus luteum formation in females, and androgen secretion in males. FSH stimulates the growth and maturation of ovarian follicles in females and spermatogenesis in males.

Both GnRH agonists and antagonists suppress gonadal steroids and decrease gonad weight. Administration of GnRH agonists is accompanied by an initial Gonadotropin and gonadal hormone surge known as flair and a consequent desensitization of GnRH-r. However suppression by GnRH is incomplete [Horvath (2002) Proc. Natl. Acad. Sci. USA 99:15048-15053]. GnRH antagonists, on the other hand, completely block and inhibit GnRH-induced GnRH receptor gene expression, leading to immediate pituitary suppression [Kovacs (2001) Proc. Natl. Acad. Sci. USA 98:1829-34]. Thus, use of GnRH antagonists with immediate suppression of the gonadal axis is therefore highly desirable.

Primary indications for GnRH antagonists include any clinical conditions in which chemical gonadotrophic hypophysectomy is required. For example treatment of cancer using GnRH antagonists is highly desirable since they exert a direct negative effect on the growth of certain malignant tumors. Several studies have shown the existence of high affinity binding sites for GnRH in human adenocarcinoma such as placenta, breast cancer, prostate cancer and ovarian cancer. In addition, inhibition of tumor cell growth by GnRH analogs has been reported [Moretti (2002) J. Clin. Endocrinol. Metab. 87:3791-3797; Tang (2002) J. Clin. Endocrinol. Metab. 87:3721-3727; Emons (1993) J. Clin. Endocrinol. Metab. 77:1458-[464]. Other indications for GnRH antagonists include, immediate blockade of the effect of gonadotropic hormones such as for fertility treatment e.g., in IVF to prevent the normal midcycle rise in LH; indirect blockade of gonadal sex-hormone secretion for treating sex-hormone dependent diseases such as benign leiomyoma and endometriosis or precocious puberty.

Though simple in theory, the development of clinically safe GnRH analogs with satisfactory efficacy has been difficult to achieve. First-generation GnRH antagonist decapeptides, such as NaI-Glu (SEQ ID NO: 38) had a limited duration of action requiring daily subcutaneous injections; solubility limitations inducing nodule formation; and a histamine response at the site of injection [Bagatell (1989). Clin. Endocrinol. Metab. 69:43-48]. By changing the peptides at position five or six, newer decapeptides were developed with fewer side effects. These include Abarelix, Acyline; Antarelix, Cetrorelix, Degarelix, Ganirelix, Iturelix, Omirelix and Antide, all being registered trade marks).

The use of currently available GnRH analogs for the treatment of cancer is limited, mainly due to the conversion of the tumor into a hormone-independent one and the loss of sensitivity to the preparation. Furthermore, clinical data on therapeutic efficacy of GnRH analogs in the treatment of gynecological cancers (e.g., ovarian, beast and endometrial cancers) is either unavailable or shows poor efficacy compared to other therapeutic modalities [e.g., treatment of breast cancer with tamoxifen; see Huirne (2001) The Lancet 358:1793-1803].

Practical cytotoxic chemotherapy-conjugated GnRH analogs which can be targeted to GnRH receptors on tumors have been synthesized and successfully tested in experimental cancer models.

The cytotoxicities of several conjugates were markedly augmented beyond that of the drug component alone. The conjugates exhibited high specific binding affinity toward the corresponding receptors, and enhanced cytotoxicity (in-vivo as well as in-vitro) to cells that express the receptors. These results further indicated that the GnRH conjugates retain their hormonal activity after administration in vivo and can apparently be bound to tumors that have receptors for GnRH.

One of the disadvantages of many currently existing analogs is the presence of histidine and/or tryptophan residues in positions number 2 and number 3 respectively (see SEQ ID NO: 1). This is due to the fact that they are subjected to collateral reactions in the course of peptide synthesis Consequently it can cause purification problems and decrease in synthesis efficiency.

The current drug-conjugated GnRH analogues, although having anti-proliferative effects on the cancer cells, are not causing cell death by themselves. This is mainly because the molecule has no direct cytotoxic action. The current existing GnRH based carrier molecules are involved in receptor mediated endocytosis process, however they don't possess the characteristics of passing across the biological membranes. The only cytotoxic effect is caused by the chemotheraputic compounds, which upon conjugation to the GnRH analogs, become targeted toxins directed specifically to the target cell, which after internalization would cause cell death.

There is thus a widely recognized need for, and it would be highly advantageous to have, peptides, useful for treating GnRH-associated diseases devoid of the above limitations.

### SUMMARY OF THE INVENTION

According to one aspect of the present invention there is provided a peptide comprising a hydrophobic moiety attached to an amino acid sequence capable of binding a GnRH receptor, the amino acid sequence being at least 11 amino acids in length.

According to another aspect of the present invention there is provided a peptide comprising a hydrophobic moiety attached to an amino acid sequence selected from the group consisting of SEQ ID NO: 2, 4, 5, 6, 7, 8, 9, 10, 12, 13, 14, 15, 16, 18, 21, 22, 23, 24, 27, 28, 32, 33, 34, 35 and 36.

According to yet another aspect of the present invention there is provided a pharmaceutical composition comprising as an active ingredient a therapeutic effective amount of a peptide including a hydrophobic moiety attached to an amino acid sequence capable of binding a GnRH receptor, the amino acid sequence being at least 11 amino acids in length.

According to still another aspect of the present invention there is provided a pharmaceutical composition comprising as an active ingredient a therapeutic effective amount of a peptide including a hydrophobic moiety attached to an amino acid sequence selected from the group consisting of SEQ ID NO: 2, 4, 5, 6, 7, 8, 9, 10, 12, 13, 14, 15, 16, 18, 21, 22, 23, 24, 27, 28, 32, 33, 34, 35 and 36.

According to an additional aspect of the present invention there is provided an article-of-manufacture comprising packaging material and a pharmaceutical composition identified for treating a GnRH associated disease being contained within the packaging material, the pharmaceutical composition including, as an active ingredient, a peptide including a hydrophobic moiety attached to an amino acid sequence capable of binding a GnRH receptor, the amino acid sequence being at least 11 amino acids in length.

According to yet an additional aspect of the present invention there is provided an article-of-manufacture comprising packaging material and a pharmaceutical composition identified for treating a GnRH associated disease being contained within the packaging material, the pharmaceutical composition including, as an active ingredient, a peptide including a hydrophobic moiety attached to an amino acid sequence selected from the group consisting of SEQ ID NO: 2, 4, 5, 6, 7, 8, 9, 10, 12, 13, 14, 15, 16, 18, 21, 22, 23, 24, 27, 28, 32, 33, 34, 35 and 36.

According to still an additional aspect of the present invention there is provided use of a peptide including a hydrophobic moiety attached to an amino acid sequence capable of binding a GnRH receptor, the amino acid sequence being at least 11 amino acids in length, for the manufacture of a medicament for the treatment and/or prevention of a GnRH associated disease.

According to a further aspect of the present invention there is provided use of a peptide including a hydrophobic moiety attached to an amino acid sequence selected from the group consisting of SEQ ID NO: 2, 4, 5, 6, 7, 8, 9, 10, 12, 13, 14, 15, 16, 18, 21, 22, 23, 24, 27, 28, 32, 33, 34, 35 and 36 for the manufacture of a medicament for the treatment and/or prevention of a GnRH associated disease.

According to yet a further aspect of the present invention there is provided a method of treating a GnRH associated disease in a subject the method comprising providing to a subject in need thereof a therapeutic effective amount of a peptide including a hydrophobic moiety attached to an amino acid sequence capable of binding a GnRH receptor, the amino acid sequence being at least 11 amino acids in length

According to still a further aspect of the present invention there is provided a use of the peptide as a pharmaceutical.

According to yet a further aspect of the present invention there is provided a use of the peptide for the manufacture of a medicament for the treatment and/or prevention of a GnRH associated disease.

According to further features in preferred embodiments of the invention described below, the amino acid sequence has a GnRH agonistic activity.

According to still further features in the described preferred embodiments the amino acid sequence has a GnRH antagonistic activity.

According to still further features in the described preferred embodiments the amino acid sequence is devoid of histidine and/or tryptophane.

According to still further features in the described preferred embodiments the hydrophobic moiety is a fatty acid.

According to still further features in the described preferred embodiments the amino acid sequence includes the following general Formula:
X₁-X₂-X₃-Ser-Tyr-X₄-Leu-Arg-Pro

According to still further features in the described preferred embodiments X₄ is an amino group containing side chain amino acid.

According to still further features in the described preferred embodiments the amino group containing side chain amino acid is an amino acid selected from the group consisting of d-Lys, d-Orn, d-Dab, d-Dap and d-Arg.

According to still further features in the described preferred embodiments the amino acid sequence further including an N-terminal amide group positioned C-terminally of the Pro.

According to still further features in the described preferred embodiments the N-terminal amide group is selected from the group consisting of Gly-NH₂, Azgly-NH₂, d-Ala-NH₂ and NH-Alkyl.

According to still further features in the described preferred embodiments the X₁ is Gly or Pro.

According to still further features in the described preferred embodiments the X₂ is an aromatic amino acid.

According to still further features in the described preferred embodiments the aromatic amino acid is Phe.

According to still further features in the described preferred embodiments the X₃ is Pro, Ala or Trp.

According to still further features in the described preferred embodiments further including an X₅ positioned N-terminally of X₁.

According to still further features in the described preferred embodiments the X₅ is selected from the group consisting of Pro and Lys.

According to still further features in the described preferred embodiments the amino acid sequence further includes X₅ positioned between X₁ and X₂.

According to still further features in the described preferred embodiments X₅ is selected from the group consisting of Pro, Lys, Gly and Ala.

According to still further features in the described preferred embodiments the amino acid sequence further includes an X₅-X₆ dipeptide positioned N-terminally of X1.

According to still further features in the described preferred embodiments the X₅ of the X₅-X₆ dipeptide is Pro or Lys.

According to still further features in the described preferred embodiments wherein X₆ of the X₅-X₆ dipeptide is selected from the group consisting of Pro, Lys, Gly and Ala.

According to still further features in the described preferred embodiments the peptide further includes a nuclear localization signal.

According to still further features in the described preferred embodiments the peptide further includes a cytotoxic agent attached thereto.

According to still further features in the described preferred embodiments the cytotoxic agent is attached to X₄.

According to still further features in the described preferred embodiments the cytotoxic agent is attached to X₅.

According to still further features in the described preferred embodiments the cytotoxic agent is attached to X₅.

According to still further features in the described preferred embodiments the cytotoxic agent is selected from the group consisting of a toxin, a radioactive isotope and a chemotherapeutic agent.

According to still further features in the described preferred embodiments the toxin is selected from the group consisting of ricin, Modeccin, Adenia digitata Abrin toxin, Abrus precatorius toxin, amanitin, pokeweed antiviral protein, gelonin, diphteria toxin, Pseudomonas serotoxin, shiga toxin and Verotoxin.

According to still further features in the described preferred embodiments the chemotherapeutic agent is selected from the group consisting of Methotrexate, 5FU, CMFU and Daunomycin Doxorubicin, 5-Fluorouracil (5FU), 1-Carboxymethyl-5-Fluorouracil (CMFU), Cytosine arabinoside (Ara-C), Cyclophosphamide, Thiotepa, Busulfan, Cytoxin, Taxol, Toxotere, Methotrexate, Cisplatin, Melphalan, Vinblastine, Bleomycin, Etoposide, Ifosfamide, Mitomycin C, Mitoxantrone, Vincreistine, Vinorelbine, Carboplatin, Teniposide, Daunomycin, Caminomycin, Aminopterin, Dactinomycin and Mitomycins.

According to still further features in the described preferred embodiments the radioactive isotope is selected from the group consisting of α-radiation emitters, β-radiation emitters and γ-radiation emitters.

According to still further features in the described preferred embodiments the amino acid sequence is as set forth by SEQ ID NO: 2, 4, 5, 6, 7, 8, 9, 10, 12, 13, 14, 15, 16, 18, 21, 22, 23, 24, 27, 28, 32, 33, 34, 35 or 36.

According to still further features in the described preferred embodiments at least one amino acid of the amino acids sequence is D stereoisomer.

According to still a further aspect of the present invention there is provided a method of treating a GnRH associated disease in a subject the method comprising providing to a subject in need thereof a therapeutic effective amount of a peptide including a hydrophobic moiety attached to an amino acid sequence selected from the group consisting of SEQ ID NO: 2, 4, 5, 6, 7, 8, 9, 10, 12, 13, 14, 15, 16, 18, 21, 22, 23, 24, 27, 28, 32, 33, 34, 35 and 36.

According to still further features in the described preferred embodiments the hydrophobic moiety is a fatty acid.

According to still further features in the described preferred embodiments the fatty acid is composed of 16-24 carbon atoms.

According to still further features in the described preferred embodiments the fatty acid is palmitic acid or lignoceric acid.

According to still further features in the described preferred embodiments the hydrophobic moiety is selected from the group consisting of an aliphatic compound, alicyclic compound and an aromatic compound.

The present invention successfully addresses the shortcomings of the presently known configurations by providing peptides which can be used to prevent and treat GnRH-associated diseases.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a photograph showing adenocarcinoma cells treated with the GnRH analogs of the present invention following a Hemacolor assay.

FIGS. 2a-c are graphs depicting the growth inhibitory effect of various concentrations of NLS-conjugated and non-conjugated GnRH analogs on adenocarcinoma cell lines as determined by the Hemacolor assay.

FIG. 3 is a photograph showing Colo-25 cells subjected to GnRH peptide analogs following a Hemacolor assay.

FIGS. 4a-c are graphs depicting the growth inhibitory effect of various concentrations of NLS-conjugated and non-conjugated GnRH analogs on adenocarcinoma cell lines as determined by the Hemacolor assay.

FIG. 5 is a photograph showing selective anti-cancerous activity of the peptides of the present invention.

### DESCRIPTION OF THE PREFERRED EMBODIMENTS

The present invention is of peptides, which can be used to prevent and treat GnRH-associated diseases, such as cancer.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Pulsalite Gonadotropin—releasing hormone (GnRH) stimulates the pituitary secretion of luteinizing hormone (LH) and follicle stimulating hormone (FSH) and thus controls the hormonal and reproductive function of the gonads.

Blockage of GnRH effects may be desired for a number of clinical conditions including prevention of untimely luteinisation during assisted reproduction or in the treatment of sex-hormone dependent disorders such as endometriosis, leiomyoma and breast cancer in women, benign prostatic hypertrophy and prostatic carcinoma in men, and central precocious puberty in children. Selective blockade of LH/FSH secretion and subsequent chemical castration has been achieved by GnRH agonists and antagonists, collectively termed GnRH analogs.

The use of currently available GnRH analogs for the treatment of cancer is limited, mainly due to the conversion of the tumor into a hormone-independent one and the loss of sensitivity to these GnRH analogs. Furthermore, clinical data on therapeutic efficacy of GnRH analogs in the treatment of gynecological cancers (e.g., ovarian, beast and endometrial cancers) is either unavailable or shows poor efficacy compared to other therapeutic modalities [e.g., treatment of breast cancer with tamoxifen; see Huirne (2001) The Lancet 358:1793-1803].

One approach to improve anti-tumor therapy based on GnRH analogs is the preparation of hybrid compounds, which consist of a GnRH decapeptide analog conjugated to a cytotoxic agent [e.g., doxorubicin (DOX), 2-pyrrolino-DOX or 5-fluorouracil (5-FU), reviewed by Schally Life Sci. (2003) 72(21):2305-20, Semko (1996) Peptides Abst. 24^{th} Symp. Eur. Pept. Soc. P26] or a palmitoyl group (Palm) which enhances anti-tumor activity of the analog [Semko (1996) Peptides Abst. 24^{th} Symp. Eur. Pept. Soc. P26]. This approach enables decreasing the doses of the cytotoxic agent and reduces risks associated with the presence of hormone-independent tumor cells.

While reducing the present invention to practice, the present inventors uncovered that palm-conjugated GnRH analogs, which are composed of at least 11 amino acids, are characterized by high cytotoxic activity when applied to GnRH expressing cells and as such can be used as valuable tools for the treatment of all current indications for GnRH analogs, especially cancer.

As is illustrated in Example 2 of the Examples section which follows, palm-conjugated GnRH analogs which include at least 11 amino acids exhibit at least 2 fold higher activity than similar decapeptide conjugates, indicating that amino acid length contributes to peptide efficacy (see e.g., Table 6 below, and compare activity of SEQ ID NO:3 to SEQ ID NOs: 2, 4-10).

Thus, according to one aspect of the present invention there is provided a peptide, which includes at least one hydrophobic moiety attached to an amino acid sequence capable of binding a GnRH receptor and being at least 11 amino acids in length.

As used herein “a GnRH receptor” refers to a cell surface protein which is able to specifically bind GnRH and analogs thereof. Examples of a GnRH receptor include GnRH type I and type II receptors which belong to the rhodopsin-like G-protein-coupled receptor family [Probst (1992) DNA Cell Biol. 11:1-20].

As used herein the phrase “hydrophobic moiety” refers to any substance which is nonpolar and generally immiscible with water.

A hydrophobic moiety according to the present invention is preferably a hydrophobic residue (portion) of a hydrophobic substance. The hydrophobic moiety of the present invention can be attached to the amino acid sequence via covalent interactions.

Representative examples of hydrophobic substances from which the hydrophobic moiety of the present invention can be derived include, but are not limited to, substituted and unsubstituted, saturated and unsaturated hydrocarbons, where the hydrocarbon can be an aliphatic, an alicyclic or an aromatic compound and preferably includes at least 4 carbon atoms, more preferably at least 8 carbon atoms, more preferably at least 10 carbon atoms, more preferably at least 12 carbon atoms, more preferably at least 16 carbon atoms, more preferably at least 24 carbon atoms. Preferably, the hydrocarbon bears a functional group which enables attachment thereof to an amino acid residue. Representative examples of such functional groups include, without limitation, a free carboxylic acid (C(═O)OH), a free amino group (NH₂), an ester group (C(═O)OR, where R is alkyl, cycloalkyl or aryl), an acyl halide group (C(═O)A, where A is fluoride, chloride, bromide or iodide), a halide (fluoride, chloride, bromide or iodide), a hydroxyl group (OH), a thiol group (SH), a nitrile group (C≡N), a free C-carbamic group (NR″—C(═O)—OR′, where each of R′ and R″ is independently hydrogen, alkyl, cycloalkyl or aryl), a free N-carbamic group (OC(═O)—NR′—, where R′ is as defined above), a thionyl group (S(═O)₂A, where A is halide as defined above) and the like.

Preferably, the hydrophobic moiety of the present invention is a fatty acid. Preferred fatty acids, which may be used in the peptides of the present invention, include saturated or unsaturated fatty acids that have more than 12 carbon atoms, preferably between 12 and 24 carbon atoms, even more preferably between 16-24 carbon atoms. Examples of fatty acids include, but are not limited to, myristic acid, lauric acid, palmitic acid, stearic acid (C18), oleic acid, linolenic acid and arachidonic acid. According to presently known configurations, palmitic acid and lignoceric acid are the preferred hydrophobic moieties according to this aspect of the present invention.

Any of the attachment approaches described hereinabove can be utilized to attach a fatty acid to the amino acid sequence of the present invention. Examples of specific approaches, which can be utilized to attach a fatty acid to an amino acid sequence are described in Example 1 of the Examples section which follows.

Alternatively, the hydrophobic moiety can be an amino acid residue that is modified to include a fatty acid residue, or any other residue of a hydrophobic substance as described hereinabove, such that this modified amino acid residue is attached to the amino acid sequence via a peptide bond or a substituted peptide bond, as is described hereinbelow. Still alternatively, the hydrophobic moiety can be a short peptide in which one or more amino acid residues are modified to include a fatty acid residue or any other residue of a hydrophobic substance as described hereinabove. Such a peptide preferably includes between 2 and 15 amino acid residues and is attached to the amino acid sequence via a peptide bond or a substituted peptide bond, as is described hereinbelow.

As an alternative to, or in combination with the hydrophobic moiety described above, the peptide of the present invention, can also include a hydrophobic peptide sequence attached to the amino acid sequence described above. This hydrophobic peptide sequence preferably includes between 2 and 15 amino acid residues, in which at least one amino acid residue is a hydrophobic amino acid residue.

Representative examples of hydrophobic amino acid residues include, without limitation, an alanine residue, a cysteine residue, an isoleucine residue, a leucine residue, a valine residue, a phenylalanine residue, a tyrosine residue, a methionine residue, a proline residue and a tryptophan residue, or any modification thereof, as is described hereinabove.

Alternatively, the hydrophobic peptide sequence can include a combination of naturally occurring and synthetic amino acids, which have been modified by incorporation of a hydrophobic moiety thereto.

The hydrophobic moiety or moieties of the present invention are preferably attached to the N-terminus and/or the C-terminus of the amino acid sequence of the peptide of the present invention.

As is mentioned hereinabove, the amino acid sequence of the peptide of the present invention is selected capable of binding a GnRH receptor. Preferably, the amino acid sequence is characterized by GnRH agonistic activity or antagonistic activity. It will be appreciated that GnRH analogs of any type (i.e., antagonists or agonists) down-regulate GnRH signaling albeit with different kinetics [see Herbst (2003) Current Opinion in Pharmacology 3:660-666]. For this reason selection of the amino acid sequence depends on the intended use of the peptide.

The amino acid sequence of the peptide of the present invention is composed of 11-13, more preferably of 11-12, most preferably of 11 amino acids.

Preferably, the amino acid sequence of the peptide of this aspect of the present invention is devoid of histidine and/or tryptophane, which may complicate synthesis and purification of the peptide [Laboratory Techniques in Biochemistry and Molecular Biology (1988), Chapt. 2 Solid-Phase Peptide Synthesis].

Preferably, the amino acid sequence of the peptide of the present invention includes the general formula:
X₁-X₂-X₃-Ser-Tyr-X₄-Leu-Arg-Pro

Wherein X₁ is preferably glycine or proline. It will be appreciated that the presence of a secondary amino group at the N-terminal proline allows for further N-terminal modification of the peptide which may facilitate the attachment of the above-described hydrophobic moiety.

X₂ is an aromatic amino acid. Examples of aromatic amino acids include, but are not limited to, phenylalanine, tyrosine and tryptophane. It is well established that the incorporation of an aromatic amino acid at this position results in peptides having a GnRH antagonistic activity [Beattie (1975) J. Med. Chem. 18(12):1247-50]. According to presently known embodiments of this aspect of the present invention, the aromatic amino acid is phenylalanine and preferably a D-stereoisomer thereof.

X₃ is proline, alanine or tryptophane [see Humphries (1978) J. Med. Chem. 21(1): 120-3].

X₄ is an amino group containing side chain amino acid, such as for example, lysine, arginine, ornithine (Orn), diaminopropionic acid (Dap) and diaminobutiric acid (Dab), for further modification of the peptide such as with a cytotoxic agent as further described hereinbelow.

The above-described amino acid sequence may further include an N-terminal amide group (Z) positioned C-terminally (downstream) of the Proline in the above-described amino acid sequence. Examples of such an N-terminal amide group include, but are not limited to, Gly-NH₂, Azgly-NH₂, d-Ala-NH₂ and NH-Alkyl.

The above-described amino acid sequence may further include proline or lysine (X₅) positioned N-terminally (upstream) of X₁. Alternatively, a proline, lysine, glycine or alanine (X₅) may be positioned between X₁ and X₂. Yet alternatively, the above-described amino acid sequence may further include a diamino acid sequence (X₅-X₆) positioned N-terminally of X₁. In this case, X₅ of the X₅-X₆ diamino acid sequence is preferably proline or lysine, while X₆ of the X₅-X₆ diamino acid sequence is preferably proline, lysine, glycine or alanine.

The peptide of the present invention may also include a nuclear localization signal of 4-7 amino acids, which actively transports the peptide, following receptor endocytosis, through nuclear envelope pores, thereby increasing activity of the peptides and especially cytotoxic conjugates thereof (further described hereinbelow, see SEQ ID NOs: 21-24). Such a nuclear localization signal may be incorporated anywhere in the peptide as long as its GnRH receptor binding activity is maintained.

Preferably, the amino acid sequence of the peptide of the present invention is as set forth in SEQ ID NO: 2, 4, 5, 6, 7, 8, 9, 10, 12, 13, 14, 15, 16, 18, 21, 22, 23, 24, 27, 28, 32, 33, 34, 35 or 36.

The term “peptide” as used herein encompasses native peptides (either degradation products, synthetically synthetic peptides or recombinant peptides) and peptidomimetics (typically, synthetic peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2—NH, CH2—S, CH2—S═O, O═C—NH, CH2—O, CH2—CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

As mentioned hereinabove, peptide bonds (—CO—NH—) within the peptide may be substituted, for example, by N-methylated bonds (—CO—N(CH3)-), ester bonds (—C(R)H—CO—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (e.g., 2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 1 and 2 below list naturally occurring amino acids (Table 1) and non-conventional or modified amino acids (e.g., synthetic, Table 2) which can be used with the present invention.

TABLE 1

| Amino Acid | Three-Letter Abbreviation | One-letter Symbol |
|---|---|---|
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| isoleucine | Iie | I |
| leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid | Xaa | X |
| as above | | |

TABLE 2

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Abgly |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methylvaline | Mval | L-α-methyltyrosine | Mtyr |
| L-α-methylleucine | Mleu | L-N-methylhomophenylalanine | Nmhphe |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| ethylamino)cyclopropane | | | |

The peptides of the present invention are preferably utilized in a linear form, although it will be appreciated that in cases where cyclization does not severely interfere with peptide characteristics, cyclic forms of the peptide can also be utilized.

Cyclic peptides can either be synthesized in a cyclic form or configured so as to assume a cyclic form under desired conditions (e.g., physiological conditions).

For example, a peptide according to the teachings of the present invention can include at least two cysteine residues flanking the core peptide sequence. In this case, cyclization can be generated via formation of S—S bonds between the two Cys residues. Side chain to side chain cyclization can also be generated via formation of an interaction bond of the formula —(—CH₂—)n—S—CH₂—C—, wherein n=1 or 2, which is possible, for example, through incorporation of Cys or homoCys and reaction of its free SH group with, e.g., bromoacetylated Lys, Om, Dab or Dap. Furthermore, cyclization can be obtained, for example, through amide bond formation, e.g., by incorporating Glu, Asp, Lys, Om, di-amino butyric (Dab) acid, di-aminopropionic (Dap) acid at various positions in the chain (—CO—NH or —NH—CO bonds). Backbone to backbone cyclization can also be obtained through incorporation of modified amino acids of the formulas H—N((CH₂)n—COOH)—C(R)H—COOH or H—N((CH₂)n—COOH)—C(R)H—NH₂, wherein n=1-4, and further wherein R is any natural or non-natural side chain of an amino acid.

The peptides according to the present invention can further include salts and chemical derivatives of the peptides. As used herein, the phrase “chemical derivative” describes a polypeptide of the invention having one or more residues chemically derivatized by reaction of a functional side group. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. Also included as chemical derivatives are those peptides that contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine. The chemical derivatization does not comprehend changes in functional groups which change one amino acid to another.

The peptides of the present invention may include useful modifications which are designed to increase the stability of the peptides in solution and, therefore, serve to prolong the half-life of the peptides in solutions, particularly biological fluids, such as blood, plasma or serum, by blocking proteolytic activity in the blood. Hence, the peptides of the present invention can have a stabilizing group at one or both termini. Typical stabilizing groups include amido, acetyl, benzyl, phenyl, tosyl, alkoxycarbonyl, alkyl carbonyl, benzyloxycarbonyl and the like end group modifications.

The amino acid sequence of the peptides of the present invention may be synthesized using any method known in the art, such as solution or solid-phase peptide synthesis, or semi-synthesis in solution beginning with protein fragments coupled through conventional solution methods, as described by Dugas et al (1981). The amino acid sequence of the peptides of the present invention can be chemically synthesized, for example, by the solid phase peptide synthesis of Merrifield et al (1964). Alternatively, the peptide of the present invention can be synthesized using standard solution methods (see, for example, Bodanszky, 1984). Newly synthesized peptides can be purified, for example, by high performance liquid chromatography (HPLC), and can be characterized using, for example, mass spectrometry or amino acid sequence analysis.

Alternatively, the amino acid sequence of the peptides of the invention can be generated using recombinant DNA technology, as long as no modified amino acids are included in the sequence. Systems for cloning and recombinant expression of peptides include various microorganisms and cells that are well known in recombinant technology. These include, for example, various strains of E. coli, Bacillus, Streptomyces, and Saccharomyces, as well as mammalian, yeast and insect cells. Suitable vectors for producing the peptides are known and available from private and public laboratories and depositories and from commercial vendors. See Sambrook et al, (1989). Recipient cells capable of expressing the gene product are then transfected. The transfected recipient cells are cultured under conditions that permit expression of the recombinant gene products, which are recovered from the culture. Host mammalian cells, such as Chinese Hamster ovary cells (CHO) or COS-1 cells, can be used. These hosts can be used in connection with poxvirus vectors, such as vaccinia or swinepox. Suitable non-pathogenic viruses that can be engineered to carry the synthetic gene into the cells of the host include poxviruses, such as vaccinia, adenovirus, retroviruses and the like. A number of such non-pathogenic viruses are commonly used for human gene therapy, and as carrier for other vaccine agents, and are known and selectable by one of skill in the art. The selection of other suitable host cells and methods for transformation, culture, amplification, screening and product production and purification can be performed by one of skill in the art by reference to known techniques [see, e.g., Gething et al, (1981) Nature 293(5834):620-625].

Once the amino acid sequence of the peptide of the present invention is provided, the hydrophobic moiety is conjugated thereto as described hereinabove.

To facilitate targeted cell-killing (e.g., cancer cell killing) and/or to improve its anti-GnRH activity, the peptide of the present invention may be conjugated to a cytotoxic agent.

As used herein, “a cytotoxic agent” refers to a substance (e.g., chemical or peptide), which is directly toxic to cells thereby preventing cell function, reproduction and/or growth. The cytotoxic agent may be incorporated anywhere on the peptide as long as its GnRH receptor binding activity is maintained. Preferably, the cytotoxic agent is attached to X₄ or to X₅.

Examples of cytotoxic agents include, but are not limited to, radio-isotopes, chemotherapeutic agents, synthetic or naturally occurring toxins, immunosuppressive agents, immunostimulating agents and enzymes.

Examples of chemotherapeutic agents include, but are not limited to, alkylating agents, folic acid antagonists, anti-metabolites of nucleic acid metabolism, antibiotics, pyrimidine analogs, 5-fluorouracil, cisplatin, purine nucleosides, amines, amino acids, triazol nucleosides, or corticosteroids. Specific examples include Adriamycin, Doxorubicin, 5-Fluorouracil (5FU), 1-Carboxymethyl-5-Fluorouracil (CMFU), Cytosine arabinoside (Ara-C), Cyclophosphamide, Thiotepa, Busulfan, Cytoxin, Taxol, Toxotere, Methotrexate, Cisplatin, Melphalan, Vinblastine, Bleomycin, Etoposide, Ifosfamide, Mitomycin C, Mitoxantrone, Vincreistine, Vinorelbine, Carboplatin, Teniposide, Daunomycin, Caminomycin, Aminopterin, Dactinomycin, Mitomycins, Esperamicins (see U.S. Pat. No. 4,675,187), Melphalan, and other related nitrogen mustards. Also included in this definition are hormonal agents that act to regulate or inhibit hormone action on tumors, such as tamoxifen and onapristone.

Examples of radio-isotopes include cytotoxic radio-isotopes such as β radiation emitters, γ emitters and α-radiation emitting materials. Examples of β radiation emitters which are useful as cytotoxic agents, include isotopes such as scandium-46, scandium-47, scandium-48, copper-67, gallium-72, gallium-73, yttrium-90, ruthenium-97, palladium-100, rhodium-101, palladium-109, samarium-153, rhenium-186, rhenium-188, rhenium-189, gold-198, radium-212 and lead-212. The most useful γ emitters are iodine-131 and indium-m 114. Other radio-isotope useful with the invention include α-radiation emitting materials such as bismuth-212, bismuth-213, and At-211 as well as positron emitters such as gallium-68 and zirconium-89.

Examples of enzymatically active toxins and fragments thereof which can be used as cytotoxic agents include diphtheria A chain toxin, non-binding active fragments of diphtheria toxin, exotoxin A chain (from Pseudomonas aeruginosa), shiga toxin, verotoxin, ricin A chain, abrin A chain toxin, modeccin A chain toxin, α-sarcin toxin, Abrus precatorius toxin, amanitin, pokeweed antiviral protein, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

Conjugates of the peptide and cytotoxic agent of the present invention are generated using any conjugation method known in the art. For example by using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bisazido compounds (such as bis-(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a 5-FU peptide conjugate can be generated as described by Semko (1996) Peptides Abst. 24^{th} Symp. Eur. Pept. Soc. P26. A ricin-peptide conjugate can be prepared as described in Vitetta et al., Science, 238: 1098 (1987). 1-isothiocyanatobenzyl-3-methyldiethylene triaminepentaacetic acid (MX-DTPA) is an exemplary chelating agent for conjugation of radionucleotide to the peptide (see WO94/11026).

As is mentioned hereinabove, one specific use for the peptides of the present invention is prevention or treatment of GnRH associated diseases.

Thus, according to another aspect of the present invention, there is provided a method of treating a GnRH associated disease in a subject. Preferred individual subjects according to the present invention are mammals such as canines, felines, ovines, porcines, equines, bovines, humans and the like.

The term “treating” refers to alleviating or diminishing a symptom associated with a GnRH associated disease. Preferably, treating cures, e.g., substantially eliminates, the symptoms associated with the GnRH associated disease.

As used herein the phrase “GnRH-associated disease” refers to a disease, which is dependent on GnRH activity for its onset or progression, or a disease in which the pathological tissue is characterized by abnormal GnRH receptor expression or activity.

Examples of GnRH-associated diseases and conditions which can treated with the peptides of the present invention include, but are not limited to, untimely luteinisation, cancer, such as prostate cancer, ovarian and breast cancer, benign hormone-dependent disorders such as endometriosis, myoma and premenstrual tension, uterine fibroids, hirsutism, cyclic auditory dysfunction, porphyria and precocious puberty in children.

The method includes providing to the subject a therapeutically effective amount of the peptide of the present invention. The peptide can be provided using any one of a variety of delivery methods. Delivery methods and suitable formulations are described hereinbelow with respect to pharmaceutical compositions.

The peptides of the present invention can be provided to an individual per se, or as part of a pharmaceutical composition where it is mixed with a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the peptide preparation, which is accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and propeities of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media (Mutter et al. (1979).

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, intranasal, or intraocular injections.

Alternately, one may administer a preparation in a local rather than systemic manner, for example, via injection of the preparation directly into a specific region of a patient's body.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. [See e.g., Fingl, et al., (1975) “The Pharmacological Basis of Therapeutics”, Ch. 1 p. 1].

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions including the preparation of the present invention formulated in a compatible pharmaceutical carrier may also be prepared, placed in an appropriate container, and labeled for treatment of an indicated condition.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Synthesis of the GnRH Analogues

Materials and Experimental Procedures

General peptide synthesis—Amino acid derivatives for peptide synthesis were purchased from Sigma Chemical Co., USA, Fisher Scientific, USA or Saxon Biochemicals GMBH, Germany. The chemicals were used according to manufacturer's instructions. Peptides were synthesized by solid-phase peptide synthesis using the BOC/Bzl strategy in a NPS-4000 semi-automatic synthesizer (Neosystem Laboratoires, France) on a MBHA resin (1 mmol/g; Sigma Chemical Co., USA) or Merrifield resin (1.2 mmol/g; Novabiochem, Swizerland). The following L amino acid derivatives were used: Nα-t-Butyloxycarbonyl-O-Benzyl-Serine, Nα-t-Butyloxycarbonyl-Proline, Nα-t-Butyloxycarbonyl-Leucine.H₂O, Nα-t-Butyloxycarbonyl-Glycine, Nα-t-Butyloxycarbonyl-N^{G}-(Mesitylene-2-sulfo)-Arginine, Nα-t-Butyloxycarbonyl-O-2,6-Dichlorobenzyl-Tyrosine. The following D amino acid derivatives were used: Nα-t-Butyloxycarbonyl-Nε-(2-Chlorobenzyloxycarbonyl)-D-Lysine, Nα-t-Butyloxycarbonyl-D-Phenylalanine, Nα-t-Butyloxycarbonyl-D-3(2-Naphthyl)-Alanine.

Peptides were recovered from the MBHA resin using trifluoromethanesulfonic acid (Fluka, Switzerland). Proper termination of the peptide synthesis reaction was validated using a ninhydrin test or isatin test for proline [Kaiser (1970) Analyt. Biochem. 34:595-598; Kaiser (1980) Analytica Chimica Acta. 118:149-151].

Trifluoroacetic acid (TFA) and solvents were prepared in accordance with requirements for solid phase peptide synthesis. Triethylamine (TEA) was distilled over KOH pellets, and then re-distilled over ninhydrin.

Table 3, below, lists the amino acid sequence and, where present, the identity of the fatty acid moiety, of GnRH peptide analogues synthesized for the present study, the corresponding, SEQ ID NO. and Example number, which describes synthesis procedure.

TABLE 3

| | | Synthesis |
|---|---|---|
| Amino acid | | procedure |
| SEQ ID NO: | Amino acid sequence of peptide | Example number |
| 1 | pGlu¹-His²-Trp³-Ser⁴-Tyr⁵-Gly⁶-Leu⁷-Arg⁸-Pro⁹-Gly¹⁰-NH₂ | Native GnRH |
| 2 | Palm-Pro-Gly-D-Phe-Pro-Ser-Tyr-D-Lys-Leu-Arg-Pro-Gly-NH₂ | 1a |
| 3 | Palm-Pro-D-2-Nal-Pro-Ser-Tyr-D-Lys-Leu-Arg-Pro-Gly-NH₂ | 1b |
| 4 | Palm-Gly-Pro-D-Phe-Pro-Ser-Tyr-D-Lys-Leu-Arg-Pro-Gly-NH₂ | 1b |
| 5 | Palm-Pro-Gly-Pro-D-Phe-Pro-Ser-Tyr-D-Lys-Leu-Arg-Pro-Gly-NH₂ | 1a |
| 6 | Palm-D-Pro-Gly-Pro-D-Phe-Pro-Ser-Tyr-D-Lys-Leu-Arg-Pro-Gly-NH₂ | 1a |
| 7 | Palm-Lys-Pro-Gly-D-Phe-Pro-Ser-Tyr-D-Lys-Leu-Arg-Pro-Gly-NH₂ | 1b |
| 8 | Palm-Lys-Gly-D-Phe-Pro-Ser-Tyr-D-Lys-Leu-Arg-Pro-Gly-NH₂ | 1b |
| 9 | Palm-D-Lys-Pro-Gly-D-Phe-Pro-Ser-Tyr-D-Lys-Leu-Arg-Pro-Gly-NH₂ | 1b |
| 10 | Palm-D-Lys-Gly-D-Phe-Pro-Ser-Tyr-D-Lys-Leu-Arg-Pro-Gly-NH₂ | 1b |
| 11 | Lau-Pro-Gly-D-Phe-Pro-Ser-Tyr-D-Lys-Leu-Arg-Pro-Gly-NH₂ | 1a |
| 12 | Palm-D-Pro-Gly-D-Phe-Pro-Ser-Tyr-D-Lys-Leu-Arg-Pro-Gly-NH₂ | 1a |
| 13 | Palm-Pro-Pro-Pro-D-Phe-Pro-Ser-Tyr-D-Lys-Leu-Arg-Pro-Gly-NH₂ | 1a |
| 14 | Palm-D-Pro-Pro-Pro-D-Phe-Pro-Ser-Tyr-D-Lys-Leu-Arg-Pro-Gly-NH₂ | 1a |
| 15 | Palm-Pro-Gly-D-Leu-Pro-Ser-Tyr-D-Lys-Leu-Arg-Pro-Gly-NH₂ | 1a |
| 16 | Palm-Pro-Gly-D-Phe-Ala-Ser-Tyr-D-Lys-Leu-Arg-Pro-Gly-NH₂ | 1a |
| 17 | Hex-Pro-Gly-D-Phe-Pro-Ser-Tyr-D-Lys-Leu-Arg-Pro-Gly-NH₂ | 1b |
| 18 | Palm-Pro-Ala-D-Phe-Pro-Ser-Tyr-D-Lys-Leu-Arg-Pro-Gly-NH₂ | 1a |
| 19 | Piv-Pro-D-Phe-Pro-Ser-Tyr-D-Lys-Leu-Arg-Pro-Gly-NH₂ | 1b |
| 20 | H-Pro-D-Phe-Pro-Ser-Tyr-D-Lys-Leu-Arg-Pro-NHEt | 1c |
| 21 | Palm-Pro-Gly-D-Phe-Pro-Ser-Tyr-D-Lys(H-Lys-Arg-Lys-Arg)-Leu- | 1d |
| | Arg-Pro-Gly-NH₂ | |
| 22 | Palm-D-Lys(CMFU)-Gly-D-Phe-Pro-Ser-Tyr-D-Lys(H-Lys-Arg-Lys- | 1a |
| | Arg)-Leu-Arg-Pro-Gly-NH₂ | |
| 23 | Palm-(H-Lys-Arg-Lys-Arg)D-Lys-Gly-D-Phe-Pro-Ser-Tyr-D-Lys-Leu- | 1a |
| | Arg-Pro-Gly-NH₂ | |
| 24 | Palm-(H-Lys-Arg-Lys-Arg)D-Lys-Gly-D-Phe-Pro-Ser-Tyr-D- | 1a |
| | Lys(CMFU)-Leu-Arg-Pro-Gly-NH₂ | |
| 25 | Palm-Pro-D-Phe-Pro-Ser-Tyr-D-Lys(CMFU)-Leu-Arg-Pro-Gly-NH₂ | |
| 26 | H-Pro-D-Phe-Pro-Ser-Tyr-D-Lys-Leu-Arg-Pro-Gly-NH₂ | 1a |
| 27 | H-Pro-Gly-D-Phe-Pro-Ser-Tyr-D-Lys-Leu-Arg-Pro-Gly-NH₂ | 1a |
| 28 | H-Gly-Pro-D-Phe-Pro-Ser-Tyr-D-Lys-Leu-Arg-Pro-Gly-NH₂ | 1a |
| 29 | Palm-Pro-D-Phe-Pro-Ser-Tyr-D-Lys-Leu-Arg-Pro-NHEt | 1c |
| 30 | H-Pro-D-Nal-Pro-Ser-Tyr-D-Lys-Leu-Arg-Pro-Gly-NH₂ | 1a |
| 31 | Lau-Pro-D-Phe-Pro-Ser-Tyr-D-Lys-Leu-Arg-Pro-Gly-NH₂ | |
| 32 | Palm-Pro-Gly-D-Phe-Pro-Ser-Tyr-D-Lys(H-Pro-Lys-Lys-Lys-Arg-Lys- | |
| | Val)-Leu-Arg-Pro-Gly-NH₂ | |
| 33 | Palm-D-Lys(H-Pro-Lys-Lys-Lys-Arg-Lys-Val)-Pro-Gly-D-Phe-Pro-Ser- | |
| | Tyr-D-Lys-Leu-Arg-Pro-Gly-NH₂ | |
| 34 | Palm-Pro-Gly-D-Phe-Pro-Ser-Tyr-D-Lys(Dox-14-O-glytaryl*)-Leu-Arg- | |
| | Pro-Gly-NH₂ | |
| 35 | Palm-Pro-Gly-D-Phe-Pro-Ser-Tyr-D-Lys(CMFU**)-Leu-Arg-Pro-Gly- | |
| | NH₂ | |
| 36 | Palm-D-Lys(Palm)-Pro-Gly-D-Phe-Pro-Ser-Tyr-D-Lys(H-Pro-Lys-Lys- | |
| | Lys-Arg-Lys-Val)-Leu-Arg-Pro-Gly-NH₂ | |

D-Nal denotes methyl glycine; pGlu denotes Pyroglutamic acid; NHEt denotes ethyl amide; D-2-Nal denotes D-3(2-Naphthyl)-Alanine.

### Example 1a Describes the Synthesis of the Peptides Set Forth in SEQ ID NOs: 2 and 11

Synthesis of these peptides was performed according to the following protocol, effected in a step-wise fashion:

1. Treatment of the resin and attachment of the first amino acid—1 g of 4-methylbenzhydrylamine resin at 1 mmole/g was placed in a reactor. After swelling of the resin in 20 ml of dimethylformamide (DMF), the resin was washed with 20 ml of DMF, treated with 20 ml of 5% TEA in DMF for 1 minute (min) and 2 mins, consecutively, washed three times with 20 ml of DMF, and once with methylene chloride. The reactor was then filled in with 0.52 g (3 mmoles) of N-t-Butyloxycarbonyl-Glycine in 15 mls of methylene chloride solution and all ingredients were mixed. After 10 mins of mixing, 0.465 ml (3 mmoles) of N,N′-diisopropyl-carbodiimide was added and mixing was continued for two hours (hrs). The last step of the first amino acid residue attachment was washing twice with 20 ml of DMF and once with methylene chloride.

2. Blocking of non-reacted amino groups—The reactor was filled with 15 ml of 10% solution of TEA in methylene chloride. Thereafter, 0.95 ml (10 mmoles) of acetic anhydride was added and mixed for 10 mins, followed by three washes with 20 ml of methylene chloride.

In the next stage, amino acid residues were added in accordance with the standard protocol for deprotection, neutralization, and condensation, as described below.

3. Deprotection—Deprotection was effected by washing with 20 ml of methylene chloride and two consecutive treatments with 20 ml of 60% TFA in methylene chloride for 1 min and 15 min, followed by two washes with 20 ml of methylene chloride without mixing that were followed by two washes for 1 min each with mixing.

4. Neutralization—Immediately following deprotection, neutralization was effected by washing with 20 ml of DMF for 1 min, followed by two treatments with 20 ml of 5% TEA in DMF for 1 min and 2 mins, and then three washes with 20 ml of DMF.

5. Condensation—Condensation by the carbodiimide method with addition of 1-hydroxybenzotriazole was effected as follows: 3 equivalents (3 mmol) of the corresponding protected amino acid derivative were dissolved in 3 ml of 1 M 1-hydroxybenzotriazole in DMF. The solution was cooled to 0° C. and 0.465 ml of N,N′-diisopropylcarbodiimide (3 mmol) were added while stirring. After 30 mins, the solution was added to the reactor.

Following condensation, peptidyl-polymer was washed twice with 20 ml of DMF and then once with 20 ml of methylene chloride.

Examination of the completeness of the coupling reaction was performed using a ninhydrin test or isatin test for proline. In cases where the presence of unreacted amino groups was detected, repeated condensation by the carbodiimide method with addition of 1-hydroxybenzotriazole was performed as described in Example 1a, step 5.

The peptidyl-polymer was divided in 0.1 mmole portions and the corresponding peptides were completed as follows:

6. Attachment of Palm-Pro or Lauryl-Pro—Palmitoyl or lauryl residues were attached to proline using the N-hydroxysuccinimidyl esters and then the corresponding amino acid derivatives were introduced as described in Example 1a, Step 5.

7. Cleavage of the peptide—Following attachment of the last amino acid residue, the resin was dried and peptide was cleaved according to the following procedure: 0.125 g of peptidyl-polymer was treated with 0.190 ml of thioanisole:ethanedithiol (2:1 v/v) and stirred for 10 mins and then the reaction flask was immersed in an ice bath. 1.25 ml of TFA was added while stirring for 10 mins, followed by slow, drop-wise addition of 0.125 ml of trifluoromethanesulfonic acid. The ice bath was removed and the reaction was continued at 20° C. for 2 hrs. The reaction mixture was diluted by addition of 100 ml of cooled diethyl ether and peptide was then removed from the polymer by washing three times with a minimal amount of TFA and precipitation using 100 ml of diethyl ether. The precipitated product was filtered, washed with ether, and dried in a vacuum, and then peptide was desalted on a Sephadex G-15 column (1.5 cm×50 cm) in 50% acetic acid.

8. Preliminary purification—Prior to final RP-HPLC purification of peptides with hydrophobic residues in step 10 below, a preliminary purification step was effected by of passage through an SPE (solid phase extraction) tube in 0.1% TFA.

9. Final purification—Final purification was effected by RP-HPLC using a System Gold Discovery C₁₈ column (25 cm×10 mm; 5 μm; Beckman, USA), that was eluted with 0.1% TFA or 0.1% H₃PO₄ in acetonitrile. Following purification, the peptide was freeze-dried.

Synthesis of Palm-ONSu

Synthesis of Palm-ONSu was effected as follows: 7.95 g (0.031 mol) of palmitic acid and 4.28 g (0.037 mol) of N-hydroxysuccinimide were dissolved in tetrahydrofuran and then the reaction flask was immersed in an ice bath. Thereafter, a solution of 6.4 g (0.031 mol) N,N′-diciclohexylcarbodiimide in tetrahydrofuran was added while cooling and stirring. The solution was washed with water and dried over Na₂SO₄. The solvent was evaporated and the product was crystallized in hexane. The yield was 5.91 g, m.p. 82-84° C.; R_{f}=0.54 (6). Detection of chromatograms was effected by carbonization in sulfuric acid. Thin layer chromatography was effected on “Silufol” films (Kavalier, Czechoslovakia). The following elutants were used: (1) 1% NH4OH—2-butanol 1:3; (2) t-butyl alcohol-acetic acid—water; (3) benzene-ethanol-ethyl acetate 3:1:1; (4) chloroform—ethanol—ethyl acetate—n-butyl alcohol—water 10:6:3:4:1; (5) n-butyl alcohol-acetic acid—pyridine—water 15:3:10:12; (6) chloroform—methanol—acetic acid 90:7:3.

Synthesis of Palm-Pro-OH

Synthesis of Palm-Pro-OH was effected as follows: 3.4 g (0.0096 mol) of Palm-ONSu were mixed with 1.1 g (0.0096 mol) of H-Pro-OH in DMF while cooling to 0° C. 2.7 ml. (0.0192 mol) of TEA was added and the solution was mixed for 24 hrs at RT.

Reaction was neutralized with H₂SO₄ and evaporated. The residue was dissolved in water and acidified to pH 4. The resultant precipitate was filtered and washed with water. The yield was 4.2 g, M. p. 53° C.; R_{f}=0.69 (3, m), 0.39 (1, m), 0.57(6, m).

### Synthesis of Lauryl-Pro-OH

Synthesis of Lauryl-Pro-OH was effected as described above in Example 1a for that of Palm-ONSu and Palm-Pro-OH. The resultant products were oils.

### Example 1b Describes the Syntheses of the Peptides Set Forth in SEQ ID NOS: 3, 7-10, 17 and 19

These peptides were prepared as described above in Example 1a, steps 1-7. Palmitoyl and pivaloyl residues were introduced directly into the peptide chain on the polymer as described below.

Introduction of Palmitoyl Residue

Following the deprotection and neutralization steps, which were effected as described in Example 1a, steps 3-4, ten equivalents of palmitic acid in a mixture of methylene chloride and dimethylsulfoxide (5:1) and ten equivalents of N,N′-diisopropyl-carbodiimide were added to the reactor. Mixing was continued for 2 hrs and after 12 hrs the resin was washed six times with methylene chloride. Steps of neutralization and condensation were effected as described in Example 1a steps 4 and 5, respectively.

To control the completeness of the introduction of palmitoyl residue, the bromphenol blue (BPB) test was effected [Krchnak V., (1998) Int. J. Pep. Prot. Res. 32:415-416].

Introduction of Pivaloyl Residue

Following the deprotection and neutralization steps, which were effected as described in Example 1a, steps 3-4, carbodiimide condensation with addition of 1-hydroxybenzotriazole was effected as described in Example 1a, step 5 using 10 equivalents of trimethylacetic acid (i.e., pivalic acid). After 12 hrs of mixing, the BPB test was still positive, hence a second neutralization step as described in Example 1a, step 4, and a second condensation were effected using carbodiimide with addition of azaoxybenzotriazole and 10 equivalents of trimethylacetic acid. This procedure was effected as described in Example 1a, step 5.

Prior to the final RP-HPLC purification of these peptides, as described in Example 1a, step 10, the SPE procedure was effected, as described in Example 1a, step 9.

### Example 1c Describes the Synthesis of the Peptide Set Forth of SEQ ID NO 20

Synthesis of SEQ ID NO: 20 was effected as follows in a step-wise fashion:

1. 0.180 g of Boc-Pro-OH (0.6 mmols) and 0.059 g (0.3 mmols) of Cs₂CO₃ were dissolved in a mixture of ethanol and water 3:1. The solution was evaporated and the cesium salt of Boc-Pro-OH was dried.

2. 0.5 g of Merrifield resin (1.2 mmol/g) was placed in a 100 ml flask. After swelling of the polymer in 10 ml of DMF, the Boc-Pro-OCs salt and 0.109 g (0.6 mmol) of 18-Crown-6 were added to the flask. The reaction mixture was stirred for 12 hrs at 50° C.

3. The resin was washed successively with 20 ml of a solution of DMF and water 9:1, 20 ml DMF, 20 ml ethanol and 20 ml diethyl ether.

4. The resin was dried and loading capacity was determined using picric acid (0.83 mmol/g).

5. The non-reacted groups were blocked by treatment with 0.106 g (0.55 mmol) of CsCOOCH₃ in DMF. The polymer was then washed as described in Example 1c, step 3 and dried as described above

6. Elongation of the peptide chain was effected by the standard methods, as described in Example 1a, steps 3-6.

7. Peptide was cleaved from the resin by treatment with 20 ml of anhydrous ethylamine at 0° C. for 4 hrs. Ethylamine was evaporated and the peptide was washed off the polymer using acetic acid. The solution was evaporated and the product was dried in a vacuum.

8. Final deprotection was effected by treatment with trifluoromethanesulfonic acid for 0.5 hr as described in Example 1a, step 7.

9. Purification of the peptide was effected by RP-HPLC as described in Example 1a, steps 8-9.

### Example 1d Describes the Synthesis of the Peptides Set Forth in SEQ ID NOS: 22, 24, 25 and 35

To the stirred solution of 0.060 g (0.043 mmol) of deprotected peptide in DMF were added 0.43 ml 0.1 N HCl and 0.020 g (0.064 mmol) CMFU-ONp. pH of the reaction mixture was adjusted to 8 with the triethylamine. Following 1 day, the solution was neutralised by 1N HCl and solvent was evaporated. Peptide was preliminary purified using low pressure chromatography on a column (25×50 mm) with Lichroprep RP-18 (40-63 μm, “Merck”). Final purification of product was carried out by RP HPLC. The fractions containing peptide were freeze-dried.

Synthesis of CMFU

A solution of 0.100 g (0.77 mmol) 5-Fluorouracil, 0.130 g (1.38 mmol) chloroacetic acid and 0.12 g (2.15 mmol) KOH in 10 ml H₂O was refluxed during 30 min. Then, cooled solution was passed through the column (25×100 mm) with Dowex 2×8 (Cl⁻) (Serva, Germany). The column was washed by distilled water till complete removal of 5-FU, then desired product was elutriated by 30% acetic acid. Solvent was removed by evaporation and residue was re-crystallized from the mixture of ethyl alcohol and diethyl ether.

Yield: 0.040 g (27%). m/z: 189 (MH⁺) (M calc. 188.09). M.P. 190-200° C.

Synthesis of CMFU-ONp

0.080 g (0.43 mmol) CMFU was added to the solution of 0.200 g (0.85 mmol) p-nitrophenyl trifluoroacetate in pyridine and resulted suspension was stirred on ice bath. After 1 hr ice bath was removed and reaction mixture was stirred at room temperature till complete dissolution. Then pyridine was removed by evaporation and residue was triturated with diethyl ether.

Yield: 0.107 g (80%). M.P. 195° C. (199° C. Pischel H., Holy A., Wagner G. Collect. Czech. Chem. Commun., 1979, V. 44, P. 1634-1641.

### Example 2

### The Effect of the GnRH Analogues of the Present Invention on Cell Viability

Cytotoxic activities of GnRH peptide analogue conjugates were screened on adenocarcinoma cell lines to which GnRH analogues have been shown to specifically bind [Nechushtan (1997) J. Biol. Chem. 272:11597-11603]. Adenocarcinoma cells were incubated with various concentrations of GnRH peptide conjugates, and cell death was monitored by measurement of the percentage of viable cells surviving treatment using the Hemacolor cell viability assay.

Materials And Experimental Procedures

Adenocarinoma Cell Lines

Adenocarcinoma cell lines utilized for the present study, the tissue source, and corresponding ATCC accession number are listed in Table 4 below. The cell lines were used for both in vitro experiments to assay the anti-tumor activity of the synthesized GnRH analogue(s) as well as for in vivo experiments involving induction of tumors in mice. These in vivo experiments will be described in Example 5.

TABLE 4

| ATCC | | |
|---|---|---|
| Accession | | |
| Number | Cell Line | Tissue source |
| HTB-22 | MCF7 | Human Breast adenocarcinoma, |
| | | bearing wild-type p53 |
| HTB-26 | MDA MB-23 1 | Human Breast adenocarcinoma, |
| | | bearing mutant p53 [Sturge et al. |
| | | J Cell Sci. 2002 115(Pt 4): |
| | | 699-711] |
| HTB-81 | DU-145 | Human Prostate |
| CRL-1435 | PC-3 | Human Prostate |
| CRL-2505 | 22RV1 | Human Prostate |
| CRL-1740 | LNCaP | Human Prostate |
| HTB-161 | OVCAR3 | Human Ovarian Carcinoma |
| | | [Leong Oncogene. 2004 23(33): |
| | | 5707-18] |
| CCL-231 | SW-48 | Human Colon |
| HTB-38 | HT-29 | Human Colon |
| CCL-222 | Colo-205 | Human Colon |
| | | [Basu Glycoconj J. 2004; 20(9): |
| | | 563-77] |
| HB-8065 | HepG2 | Human Hepatocarcinoma |

Growth and Expansion of Cell Lines

Cell lines were grown in tissue culture using DMEM or RPMI media supplemented with 10% FCS, 2 mM L-Glutamine, 100 units/ml Penicillin, and 100 μg/ml Streptomycin. Cells were grown as monolayers in a controlled atmosphere in a humidified incubator (37° C., 5% CO₂). Cells were expanded twice a week as follows: Media was removed from the flask and the flask was rinsed with PBS. Cells were detached by addition and incubation of 3 ml of 0.25% Trypsin-EDTA solution for 3-4 mins at 37° C. After incubation, 50 ml of medium was carefully added to the solution of detached cells in Trypsin-EDTA, and the cell-containing mixture was split into 2 flasks.

Storage of Cell Lines

Cells were removed from the flasks with Trypsin-EDTA as described above and spun at 1200 rpm for 5 min. Trypsin was removed and freezing medium (90% FCS and 10% DMSO) was added. The freezing tube was wrapped in tissue paper and stored at −80° C. for one week prior to transfer to liquid nitrogen.

Hemacolor Assay

The Hemacolor assay, a colorimetric microtiter assay that quantifies the amount of dye that binds to viable adherent cells (Keisari, Y. A colorimetric microtiter assay for the quantitation of cytokine activity on adherent cells in tissue culture. J. Immunol. Methods 146, 155-161; 1992), was effected as follows: Cells were plated at 10⁴ cells/well in flat-bottom 96 well plates in 200 μl of DMEM medium at day 0 and incubated at 37° C. overnight (o/n) in a humidified CO₂ incubator. On day 1, supernatant was removed and various concentrations of peptide were added. Cell destruction was followed under the microscope. Supernatant was carefully removed by vacuum and cells were fixed by incubation with absolute methanol at 70 μl/well for 30 seconds and then methanol was removed by vacuum. Cells were stained with Hemacolor reagents (Merck, Darmstadt, Germany) in a step-wise fashion as follows: 100 μl of Hemacolor reagent number 2 (acidic red solution, for nuclei staining) was added to the cells and was removed after 1 min by vacuum. 100 μl of Hemacolor reagent number 3 (basic blue solution, for membrane staining) was then added to the cells and removed after 1 min by vacuum. The plate was extensively rinsed and refilled with water to destain for 5 min. The plate was dried and stored for monitoring results. Dye was extracted from the plate by addition of 0.5% SDS in PBS (100 μl) with vigorous mixing. Plates were quantified at 650 nm in an ELISA reader. Cell viability of ≧95% by Trypan blue exclusion from 400 cells was a prerequisite for each experiment.

Results

Activity Screening of GnRH Peptides by Hemacolor Assay

Results of activity of the peptide are summarized in Table 5 and in Table 6.

Table 5, below, summmarizes activities of GnRH peptide analogues used in the present study as monitored by the Hemacolor Assay. Amino acid sequence using the three letter code, the corresponding SEQ ID NO, concentrations of peptide utilized, with the active concentration(s) highlighted in bold, and intensity of staining are noted.

TABLE 5

| SEQ ID | | | Intensity of |
|---|---|---|---|
| NO: | Amino Acid Sequence of Peptide | Activity | Staining |
| 3 | Palm-Pro-D-2Nal-Pro-Ser-Tyr-D-Lys-Leu-Arg-Pro- | 10⁻⁴ 5 × 10⁻⁵ 10⁻⁵ 10⁻⁶ | +++ |
| | Gly-NH₂ | | |
| 2 | Palm-Pro-Gly-D-Phe-Pro-Ser-Tyr-D-Lys-Leu-Arg- | 10⁻⁴ 5 × 10⁻⁵ 10⁻⁵ 10⁻⁶ | +++++ |
| | Pro-Gly-NH₂ | | |
| 4 | Palm-Gly-Pro-D-Phe-Pro-Ser-Tyr-D-Lys-Leu-Arg- | 10⁻⁴ 5 × 10⁻⁵ 10⁻⁵ 10⁻⁶ | ++ |
| | Pro-Gly-NH₂ | | |
| 25 | Palm-Pro-D-Phe-Pro-Ser-Tyr-D-Lys(CMFU)-Leu- | 10⁻⁴ 5 × 10⁻⁵ 10⁻⁵ 10⁻⁶ | + |
| | Arg-Pro-Gly-NH₂ | | |
| 26 | H-Pro-D-Phe-Pro-Ser-Tyr-D-Lys-Leu-Arg-Pro-Gly- | 10⁻⁴ 5 × 10⁻⁵ 10⁻⁵ 10⁻⁶ | − |
| | NH₂ | | |
| 27 | H-Pro-Gly-D-Phe-Pro-Ser-Tyr-D-Lys-Leu-Arg-Pro- | 10⁻⁴ 5 × 10⁻⁵ 10⁻⁵ 10⁻⁶ | − |
| | Gly-NH₂ | | |
| 19 | Piv-Pro-D-Phe-Pro-Ser-Tyr-D-Lys-Leu-Arg-Pro- | 10⁻⁴ 5 × 10⁻⁵ 10⁻⁵ 10⁻⁶ | − |
| | Gly-NH₂ | | |
| 28 | H-Gly-Pro-D-Phe-Pro-Ser-Tyr-D-Lys-Leu-Arg-Pro- | 10⁻⁴ 5 × 10⁻⁵ 10⁻⁵ 10⁻⁶ | − |
| | Gly-NH₂ | | |
| 29 | H-Pro-D-Phe-Pro-Ser-Tyr-D-Lys-Leu-Arg-Pro- | 10⁻⁴ 5 × 10⁻⁵ 10⁻⁵ 10⁻⁶ | − |
| | NHEt | | |
| 30 | H-Pro-D-2Nal-Pro-Ser-Tyr-D-Lys-Leu-Arg-Pro- | 10⁻⁴ 5 × 10⁻⁵ 10⁻⁵ 10⁻⁶ | − |
| | Gly-NH₂ | | |
| 31 | Lauryl-Pro-D-Phe-Pro-Ser-Tyr-D-Lys-Leu-Arg-Pro- | 10⁻⁴ 5 × 10⁻⁵ 10⁻⁵ 10⁻⁶ | − |
| | Gly-NH₂ | | |
| 1 | pGlu-His-Trp-Ser-Tyr-Gly-Leu-Arg-Pro-Gly-NH₂ | 10⁻⁴ 5 × 10⁻⁵ 10⁻⁵ 10⁻⁶ | − |
| | (GnRH) | | |

Results are expressed as mean values plus or minus

Table 6, below, summarizes activities as assessed by the Hemacolor assay of the Palm-GnRH peptide family used in the present study, and lists the length of the amino acid sequence, the name of the analogue, the corresponding SEQ ID NO, and displays the identity of a fatty acid moiety and amino acid residues using the single letter code at each position in the peptide chain relative to the sequence of native GnRH.

TABLE 6

| | number | SEQ/ | Fatty | | | | | | | | | | | | |
|---|---|---|---|---|---|---|---|---|---|---|---|---|---|---|---|
| Activity | AA | ID | Acid | −1 | 0 | 1 | 2 | 3 | 4 | 5 | 6 | 7 | 8 | 9 | 10 |
| − | 10 | 1 | | | | pGlu | His | Trp | Ser | Tyr | Gly | Leu | Arg | Pro | Gly-NH₂ |
| ++++ | 11 | 2 | Palm | | Pro | Gly | D-Phe | Pro | Ser | Tyr | D-Lys | Leu | Arg | Pro | Gly-NH₂ |
| + | 10 | 3 | Palm | | | Pro | D-2Nal | Pro | Ser | Tyr | D-Lys | Leu | Arg | Pro | Gly-NH₂ |
| +++ | 11 | 4 | Palm | | Gly | Pro | D-Phe | Pro | Ser | Tyr | D-Lys | Leu | Arg | Pro | Gly-NH₂ |
| ++ | 12 | 5 | Palm | Pro | Gly | Pro | D-Phe | Pro | Ser | Tyr | D-Lys | Leu | Arg | Pro | Gly-NH₂ |
| ++ | 12 | 6 | Palm | D-Pro | Gly | Pro | D-Phe | Pro | Ser | Tyr | D-Lys | Leu | Arg | Pro | Gly-NH₂ |
| ++ | 12 | 7 | Palm | Lys | Pro | Gly | D-Phe | Pro | Ser | Tyr | D-Lys | Leu | Arg | Pro | Gly-NH₂ |
| +++ | 11 | 8 | Palm | | Lys | Gly | D-Phe | Pro | Ser | Tyr | D-Lys | Leu | Arg | Pro | Gly-NH₂ |
| ++++ | 12 | 9 | Palm | D-Lys | Pro | Gly | D-Phe | Pro | Ser | Tyr | D-Lys | Leu | Arg | Pro | Gly-NH₂ |
| ++++ | 11 | 10 | Palm | | D-Lys | Gly | D-Phe | Pro | Ser | Tyr | D-Lys | Leu | Arg | Pro | Gly-NH₂ |
| − | 11 | 11 | Lau | | Pro | Gly | D-Phe | Pro | Ser | Tyr | D-Lys | Leu | Arg | Pro | Gly-NH₂ |
| ++ | 11 | 12 | Palm | | D-Pro | Gly | D-Phe | Pro | Ser | Tyr | D-Lys | Leu | Arg | Pro | Gly-NH₂ |
| ++ | 12 | 13 | Palm | Pro | Pro | Pro | D-Phe | Pro | Ser | Tyr | D-Lys | Leu | Arg | Pro | Gly-NH₂ |
| ++ | 12 | 14 | Palm | D-Pro | Pro | Pro | D-Phe | Pro | Ser | Tyr | D-Lys | Leu | Arg | Pro | Gly-NH₂ |
| ++ | 11 | 15 | Palm | | Pro | Gly | D-Leu | Pro | Ser | Tyr | D-Lys | Leu | Arg | Pro | Gly-NH₂ |
| ++++ | 11 | 16 | Palm | | Pro | Gly | D-Phe | Ala | Ser | Tyr | D-Lys | Leu | Arg | Pro | Gly-NH₂ |
| − | 11 | 17 | Hex | | Pro | Gly | D-Phe | Pro | Ser | Tyr | D-Lys | Leu | Arg | Pro | Gly-NH₂ |
| ++ | 11 | 18 | Palm | | Pro | Ala | D-Phe | Pro | Ser | Tyr | D-Lys | Leu | Arg | Pro | Gly-NH₂ |

### Example 3

### Further Assessment of the Effect of the GnRH Analogues of the Present Invention on Cell Viability

For a second assessment of anti-tumor activity of GnRH peptide analogues, the MTT assay may be performed.

Materials and Experimental Procedures

MTT Assay

The MTT assay is a functional assay that measures cell viability via mitochondrial activity of viable cells. The mitochondrial enzyme succinate dehydrogenase of viable cells causes the formation (reduction) of blue formasan crystals from yellow-colored MTT substrate (or Bromure of 3-(4,5-dimethylthiazol-2-yl)-2,5-diphenyl tetrazolium bromide, which, following dissolution of the crystals, can be measured by a spectrophotometric reading, giving an optical density that is directly proportional to the number of viable cells (Mossman, T. Rapid colorimetric assay for cellular growth and survival: application to proliferation and cytotoxicity assays. J. Immunol. Methods 65, 55-63; 1983).

The MTT assay is effected as follows: On day 0, 10⁴ cells/well of adenocarcinoma or control cells (e.g. fibroblasts) are plated in flat-bottomed 96 well plates in 200 μl of DMEM medium and plates are incubated at 37° C. for over-night in a humidified 5% CO₂ incubator. On day 1, the supernatant is removed and various concentrations of peptide are added in DMEM or RPMI medium (dependent on the cells, which are being used) and incubated for 48 h. Cell destruction is monitored under the microscope. Following incubation, 1 mg/ml of MTT solution in 100 μl PBS is added and the cells are incubated for 2-4 hrs, depending on the rate of appearance of blue color. To dissolve MTT-formasan precipitates, supernatant is carefully removed from the wells by vacuum and the cells are dissolved by addition of 100 μl acidified isopropyl alcohol (0.04 N HCl in 2-propanol). Plates are read at 560 nm on an ELISA reader to quantitate the color change. Corrections for non-specific absorption of MTT are made by subtracting background values generated in blank (i.e., cell-free) wells, containing medium, the corresponding dose of drug, and MTT solution.

### Example 4

Anti-Proliferative Effect of the GnRH Analogues of the Present Invention

The affect of GnRH peptide conjugates on cell proliferation of adenocarcinoma cell lines may be determined by the [³H]-thymidine incorporation assay. [³H]-thymidine incorporation into cellular DNA provides a measurement of DNA multiplication and cell division for assessment of DNA synthesis.

Materials and Experimental Procedures

[³H]-Thymidine Assay

Cells are plated in triplicates in flat-bottom 96 well plates at 10³ cells/well in 200 μl of DMEM at day 0 and incubated at 37° C. for over-night in a humidified CO₂ incubator. Following attachment of the cells to the surface, screened peptides are added and incubated for 24 hrs. At 20 hrs, [³H]-thymidine is added to the cells at 1 μCi/well and the incubation is continued for 4 hours. The plates are then placed at 4° C. for 24-48 hrs to terminate the reaction. Cellular DNA is harvested using a Mach III harvester and the samples are air dried for several hours. The samples are then immersed in liquid scintillation solution and radioactivity is counted in a β counter and data is plotted on a graph.

### Example 5

### Anti Adenocarcinoma Activity Induced by GnRH Peptides on Various Adenoarcinoma Cell Lines

The growth inhibitory activity of GnRH peptides generated according to the teachings of the present invention was examined on various adenocarcinoma cell lines.

Materials and Experimental Procedures

Cell lines—The following cancer cell lines were examined (ATCC ACCESSION No. are indicated)—Colon origin: HT-29 (HTB-38); SW-480 (CCL-231) Colon origin; HCT 116 (ATCC: CCL-247) Colon origin; Colo-205 (ATCC: CCL-222); Colon origin: PANC1 (ATCC: CRL 1469) Prostate orgin; DU145(ATCC:HTB-81) Prostate orgin; Mia Pacca-2 (ATCC: CRL 1420); Prostate origin; HepG2(ATCC HB-8065); Hepatocarcinoma, Liver origin: MDA-MB-231(ATCC: HTB-26) Breast origin; MCF7 (HTB-22) Breast origin.

Culturing conditions (also described in page 36 under Growth and expansion of cell lines): Cell lines were grown in tissue culture using DMEM or RPMI media supplemented with 10% FCS, 2 mM L-Glutamine, 100 units/ml Penicillin, and 100 μg/ml Streptomycin. Cells were grown as monolayers in a controlled atmosphere in a humidified incubator (37° C., 5% CO₂). Cells were expanded twice a week as follows: Media was removed from the flask and the flask was rinsed with PBS. Cells were detached by addition and incubation of 3 ml of 0.25% Trypsin-EDTA solution for 3-4 mins at 37° C. After incubation, 50 ml of medium was carefully added to the solution of detached cells in Trypsin-EDTA, and the cell-containing mixture was split into 2-4 flasks.

Hemacolor Assay—The assay is described in length in Example 2 above. Briefly, cells (10⁴ cells/well) were plated at in flat-bottom 96 well plates in 100 μl of DMEM medium at day 0 and incubated at 37° C. overnight in a humidified CO₂ incubator. Following 24 hours various concentrations (see Table 5, below) of peptide X (SEQ ID NO: 2), peptide X-5FU (5-Fluorouracil) (SEQ ID NO: 35), and peptide F (SEQ ID NO: 10) were added in total 100 μl medium. Cell destruction was followed under the microscope. Supernatant was carefully removed by vacuum aspiration and cells were fixed by incubation with absolute methanol (also termed Hemacolor reagent number 1) at 70 μl/well for 30 seconds after which methanol was removed by vacuum aspiration. Viable cells were quantified by Hemacolor assay. This calorimetric microtiter assay quantifies the amount of dye that binds to viable adherent cells (Keisari, Y. A colorimetric microtiter assay for the quantitation of cytokine activity on adherent cells in tissue culture. J. Immunol. Methods 146, 155-161; 1992). Cells were stained with Hemacolor reagents (Merck, KGaA, 64271, Darmstadt, Germany) in a step-wise manner as follows: 100 μl of Hemacolor reagent number 2 (acidic red solution No. 1.11956.2500, for nuclei staining) was added to the wells and was removed after 1 min by vacuum aspiration. 100 μl of Hemacolor reagent number 3 (basic blue solution No. 1.11957.2500, for membrane staining) was then added to the cells and removed after 1 min by vacuum aspiration. The plate was extensively rinsed and refilled with water to distain for 5 min. The plate was dried and stored for monitoring results.

Results

TABLE 7
Summary of peptides destruction of 10 adenocarcinoma cell lines

| Cell lines and | Peptide Concentration (M) | | | | | Peptide/ | Grade of cell |
|---|---|---|---|---|---|---|---|
| tumor origin | 1 × 10⁻⁴ | 5 × 10⁻⁵ | 1 × 10⁻⁵ | 5 × 10⁻⁶ | 1 × 10⁻⁶ | SEQ ID NO. | destruction |
| Colo-205 | − | − | + | + | + | 2 | A |
| colon | − | − | + | + | + | 35 | |
| | − | − | + | + | + | 10 | |
| | + | + | + | + | + | Medium | |
| DU 145 | − | − | + | + | + | 2 | A |
| prostate | − | − | + | + | + | 35 | |
| | − | − | + | + | + | 10 | |
| | + | + | + | + | + | Medium | |
| MDA-MB-231 | − | − | + | + | + | 2 | A |
| Breast | − | − | + | + | + | 35 | |
| | − | − | + | + | + | 10 | |
| | + | + | + | + | + | Medium | |
| Mia Pacca | − | − | + | + | + | 2 | A |
| Prostate | − | − | + | + | + | 35 | |
| | − | − | + | + | + | 10 | |
| | + | + | + | + | + | Medium | |
| HCT-116 | − | − | − | + | + | 2 | A |
| Colon | − | − | + | + | + | 35 | |
| | − | − | + | + | + | 10 | |
| | + | + | + | + | + | Medium | |
| HepG2 | − | − | − | + | + | 2 | A |
| Liver | − | − | + | + | + | 35 | |
| | − | − | + | + | + | 10 | |
| | + | + | + | + | + | Medium | |
| PANC-1 | − | − | + | + | + | 2 | B |
| Prostate | − | + | + | + | + | 35 | |
| | − | + | + | + | + | 10 | |
| | + | + | + | + | + | Medium | |
| HT-29 | − | − | + | + | + | 2 | B |
| Colon | − | −/+ | + | + | + | 35 | |
| | −/+ | −/+ | + | + | + | 10 | |
| | + | + | + | + | + | Medium | |
| MCF7 | − | − | + | + | + | 2 | C |
| Breast | − | −/+ | + | + | + | 35 | |
| | −/+ | −/+ | + | + | + | 10 | |
| | + | + | + | + | + | Medium | |
| SW-480 | − | −/+ | + | + | + | 2 | C |
| Colon | −/+ | −/+ | + | + | + | 35 | |
| | − | − | + | + | + | 10 | |
| | + | + | + | + | + | Medium | |
| Human | −/++ | −/+++ | + | + | + | 2 | D |
| fibroblasts | −/++ | −/+++ | + | + | + | 35 | |
| | −/++ | −/+++ | + | + | + | 10 | |
| | + | + | + | + | + | Medium | |

As is evident from Table 5 above, all adenocarinoma lines were destroyed by peptides X, F, and X-5FU (SEQ ID NOs. 2, 10 and 35, respectively) to various extents as follows:

Grade A: Colo-205; DU-145; MA-MB-231; Mia-Pacca; HCT-116; HepG2;

Grade B: PANC-1; HT-29;

Grade C: MCF-7; SW-480;

Grade D: human foreskin fibroblasts (ATCC # CRL-2429);

### Example 6

### Synthesis of Palm-NLS-GnRH Analogs and Doxorubicin Conjugate and Growth Inhibitory Activities Thereof

Nuclear targeting sequence (NLS) and chemotherapy conjugated peptides were synthesized to improve inhibitory activity of the GnRH peptide analogs of the present invention.

NLS conjugated peptides, are expected to be of improves efficacy by (withour being bound by theory) interacting with specific receptors at the nuclear pores. These peptides were later analyzed on three adenocarcinoma cell lines: MDA-MB-231(breast) HCT-116 and Colo-205 (colon).

Materials and Experimental Procedures

The following peptides were synthesized:

Peptide X (SEQ ID NO: 2, number 4 of Table 8 below) conjugated to the sequence: H-Pro-Lys-Lys-Lys-Arg-Lys-Val (SEQ ID NO: 37). This results in NLS-X (SEQ ID NO: 32, number 5 of Table 8 below).

Synthesis Procedures—Peptide E (SEQ ID NO: 9, number 1 of Table 8 below) conjugated to the sequence: H-Pro-Lys-Lys-Lys-Arg-Lys-Val, resulting in NLS-E (SEQ ID NO: 33, number 6 of Table 8 below).

1. Peptide E (SEQ ID NO: 9, number 1 of Table 8 below) conjugated to the sequence: H-Pro-Lys-Lys-Lys-Arg-Lys-Val, and an additional moiety of Palm, resulting in (Palm2-NLS-E) NLSp2 (SEQ ID NO: 36 number 9 of Table 8 below).

2. Peptide X conjugated to Doxorubicin (SEQ ID NO: 34 No. 7 of Table 8 below).

3. Derivatives for DOX-X synthesis were prepared as described by Schally (Life Sci. (2003) 72(21):2305-20).

Synthesis of peptides NLS-E, NLS-X and 2pNLS-X (SEQ ID NO 32, 33 and 36)—Synthesis of these peptides was performed according to the protocol described in Example 1a, steps 1-5 till the point of NLS moiety attachment.

Fmoc-D-Lys(Boc)-OH was attached by the standard methodology, and then NLS sequence was accomplished using Boc—protocol (Example 1a, steps 3-5), using 5% DIPEA in DMF at the step 4 (Neutralisation).

Then synthesis was accomplished using Fmoc—protocol:

1. Deprotection—deprotection was effected by washing with 20 ml of DMF and two consecutive treatments with 20 ml of 20% piperidine in DMF for 1 min and 8 min, followed by three washes with 20 ml of DMF, three washes with 20 ml of i-PrOH and three washes with 20 ml of DMF for 1 min each of them.

2. Condensation were effected as described in Example 1a, steps 5.

3. In the case of NLS-E and NLS-X the palmitoyl moiety was introduced into peptide structure on polymer after removing of Fmoc—protection (Example 1d, stepl) as described in Example 1b.

4. In the case of 2pNLS-X, 3 eqv of the 2 Palm-D-Lys-OH were dissolved in the mixture of chloroform and phenol 3:1, in an ice bath, then 3 eqv of DIC and 6-Chloro-1-hydroxibenzotriazol were added. Reaction mixture was stirred for 30 min and then added to deprotected peptidylpolymer (Example 1d step 1) just after washing by the mixture of chloroform and phenol 3:1. Then condensation was complete, peptidylpolymer was washed by the mixture of chloroform and phenol 3:1, 3 times by chloroform, 3 times by DMF and 3 times by chloroform for 1 min each of them.

4. Cleavage of peptides was effected by trifluoromethanesulfonic acid treatment for 0.5 hr as described in Example 1a, step 7.

5. Purification of peptides was effected by RP-HPLC as described in Example 1a, steps 8-9.

Synthesis of 2 Palm-D-L s-OH—To the stirred solution of 0.5 g H-D-Lys-OH (0.0034 mmol) in DMF was added 5 g (0.014 mmol) Palm-ONSu and TEA to maintain pH 8.5. Reaction mixture was stirred overnight, neutralised by 1 N HCl, and evaporated. The residual oil was triturated in methylene chloride, then thin precipitate was filtered and washed by 1 N HCl, water, acetone and diethyl ether. Yield: 1.2 g, R_{f}=0.29 (3, m).

Synthesis of peptide DOX-X (SEQ ID NO 34, described in Example 1e, above)—Briefly, 45 mg of peptide X (31 mmol) and 35 mg N-Fmoc-DOX-14-O-hemiglutarate (31 mmol) were dissolved in 0.5 ml of DMF, and 17 mg of BOP reagent (38 mmol), 10 mg 1-hydroxybenzotriazole (76 mmol) and 42 μl of DIPEA were added. After stirring for 1 hr, the solvent was evaporated and the residual oil was crystallised in ethyl acetate and washed by decantation. The product was dissolved in 10% piperidine in DMF. After 5 min, the reaction mixture was placed into an ice bath and acidified by the addition of mixture containing 200 μl of TFA, 460 μl of pyridine and 1330 μl of DMF. The solvents were evaporated and the residual oil was crystallised in ethyl acetate and washed by decantation. The product was purified by the passage through an SPE (solid phase extraction) tube in 0.1% TFA/15% acetonitrile followed by the stepwise elution and freeze-dried from 15% acetonitrile. Synthesis of N-Fmoc-DOX-14-O-hemiglutarate-DOX HCL salt (50 mg, 86 μmol) was dissolved in 1 ml of DMF, and Fmoc-ONSu (30 mg, 90 μmol) was added, followed by 30 μl (172μ) of DIPEA. After 3 r, the solvent was evaporated and residue was crystallised from 0.1% TFA, filtered and quickly washed with cold diethyl ether. This intermediate (62 mg, yield 94%) was reacted overnight with glutaric anhydride (11.4 mg 100 μmol) in 1 ml of DMF in the presence of DIPEA (26.1 μl, 150 μmol). The solvents were evaporated and the residual oil was solidified by the water. The product was purified on a column (3×10 cm) with LiChroprep RP-18 (Merck) in 0.1% TFA/10% i-PrOH. Then final product, in small parts, was loaded on the column in pure acetonitrile with following washing by the starting buffer. Then stepwise elution was used for the purification.

Hemacolor assay—A comparative study was effected between peptides GnRH—X and NLS-GnRH-E on three adenocarcinoma cell lines: MDA-MB-231 (breast) HCT-116 and Colo-205 (colon), described in Example 5 above.

Briefly, cells were plated in 96 well plates and allowed to adhere to the plates. Thereafter, various concentrations of GnRH and NLS-GnRH peptides were incubated with the adhered cells for 24 h. To quantify the extent of damage to the cells by the peptides, the Hemacolor assay was effected as described in Example 5. Viable cells which remained adhered to the 96 plastic plates were fixed and stained. Dye was extracted from the plate by addition of 0.5% SDS in PBS (100 μl) with vigorous mixing. Plates were quantified at 650 nm by spectrophotometer (ELISA reader). Cell viability of ≧95% by Trypan blue exclusion from 400 cells was a prerequisite for each experiment. Note, the color intensity is proportional to the extent of the remaining viability.

Data was calculated as EC₅₀, which is defined as the concentration (in μM) of analog that provokes a response half way between the baseline (Bottom=control) and maximum response (Top). Lower ECso values indicate a more potent analog.

Results

TABLE 8
Peptides structures and molecular weights

| Peptide/SEQ ID NO. | | | Structure | MW |
|---|---|---|---|---|
| 1. | E | 9 | [(Palm-D-Lys-Pro-Gly)¹; D-Phe²; Pro³; D-Lys⁶]-LH-RH; | 1584.92 |
| | | | C₈₀H₁₃₀N₁₈O₁₅ | |
| 2. | F | 10 | [(Palm-D-Lys-Gly)¹; D-Phe²; Pro³; D-Lys⁶]-LH-RH; | 1486.92 |
| | | | C₇₅H₁₂₃N₁₇O₁₄ | |
| 3. | L | 16 | [(Palm-Pro-Gly)¹; D-Phe²; Ala³; D-Lys⁶]-LH-RH; | 1429.83 |
| | | | C₇₂H₁₁₆N₁₆O₁₄ | |
| 4. | X | 2 | [(Palm-Pro-Gly)¹; D-Phe²; Pro³; D-Lys⁶]-LH-RH; | 1455.86 |
| | | | C₇₄H₁₁₈N₁₆O₁₄ | |
| 5. | NLS-X | 32 | [(Palm-Pro-Gly)¹; D-Phe²; Pro³; D-Lys(H-Pro-Lys-Lys- | 2321.01 |
| | | | Lys-Arg-Lys-Val)⁶]-LH-RH; C₁₁₄H₁₉₄N₃₀O₂₁ | |
| 6. | NLS-E | 33 | [(Palm-D-Lys(H-Pro-Lys-Lys-Lys-Arg-Lys-Val)Pro-Gly)¹; | 2449.18 |
| | | | D-Phe²; Pro³; D-Lys⁶]-LH-RH; C₁₂₀H₂₀₆N₃₂O₂₂ | |
| 7. | X-DOX | 34 | [(Palm-Pro-Gly)¹; D-Phe²; Pro³; D-Lys(Dox*)⁶]-LH-RH; | 2095.48 |
| | | | C₁₀₆H₁₅₁N₁₇O₂₇ | |
| 8. | X-5FU | 35 | [(Palm-Pro-Gly)¹; D-Phe²; Pro³; D-Lys(CMFU**)⁶]-LH-RH; | |
| | | | C80H121N18O17F1 | |
| 9. | (Palm2-NLS-E) | 36 | [(Palm-D-Lys(Palm)-Pro-Gly)¹; D-Phe²; Pro³; D-Lys(H- | 2687.60 |
| | NLSp2 | | Pro-Lys-Lys-Lys-Arg-Lys-Val)⁶]-LH-RH; C₁₃₆H₂₃₆N₃₂O₂₃ | |

*Doxorubicin
**5-FluoroUracil (5FU)

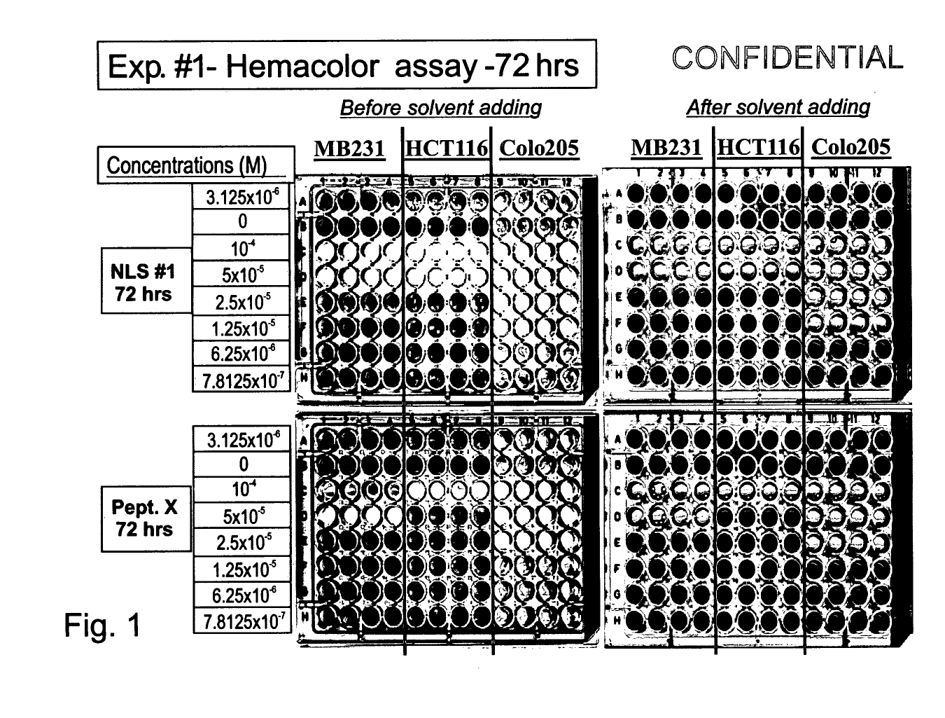

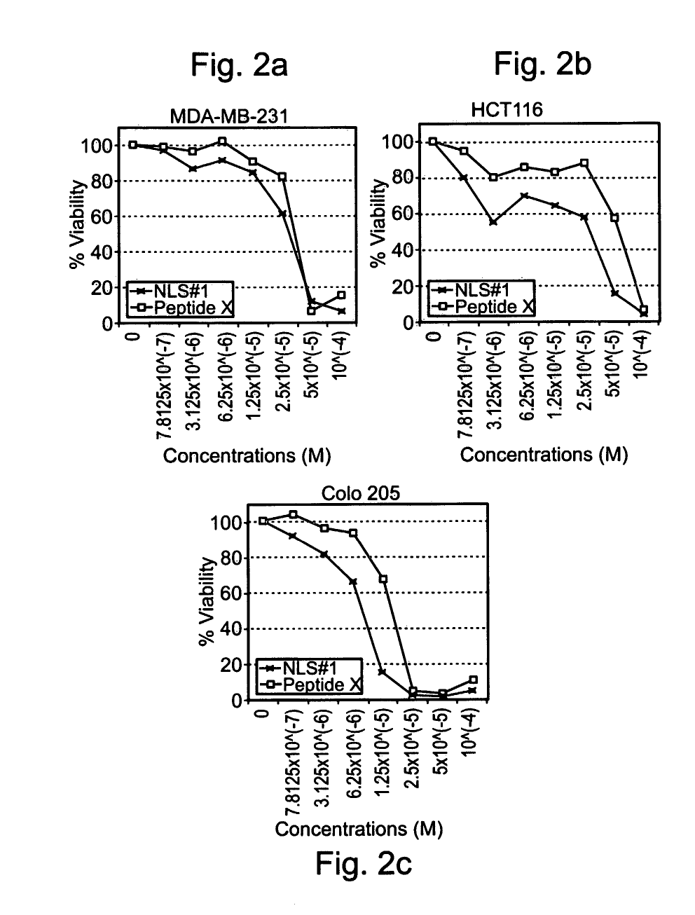

The EC₅₀ values of NLS-E and X peptides activity on 3 adenocarcinoma cell lines (Table III) was calculated from the results of the hemacolor test (FIGS. 1 and 2a-c).

The results are summarized in Table 9 below.

TABLE 9

EC₅₀ (μM)

| Adenocarcinoma Line | NLS-E | X |
|---|---|---|
| COLO-205 | 68 | 200 |
| MDA-MB-231 | 350 | 400 |
| HCT-116 | 320 | 750 |

Evidently, the best responder adenocarcinoma cell line was Colo-205. As expected NLS conjugated peptides (NLS-E) exhibited better efficacy than NLS free peptide (X).

### Example 7

### Growth Inhibitory Effect of Palm-GnRH Peptides Versus NLS-Palm-GnRH Peptides as Determined on Colo-205 Adenocarcioma Cell Line

Materials and Experimental Procedures

Peptides were synthesized as described in Example 6 above. Growth inhibitory activity was demonstrated by the Hemacolor assay, described hereinabove.

Results

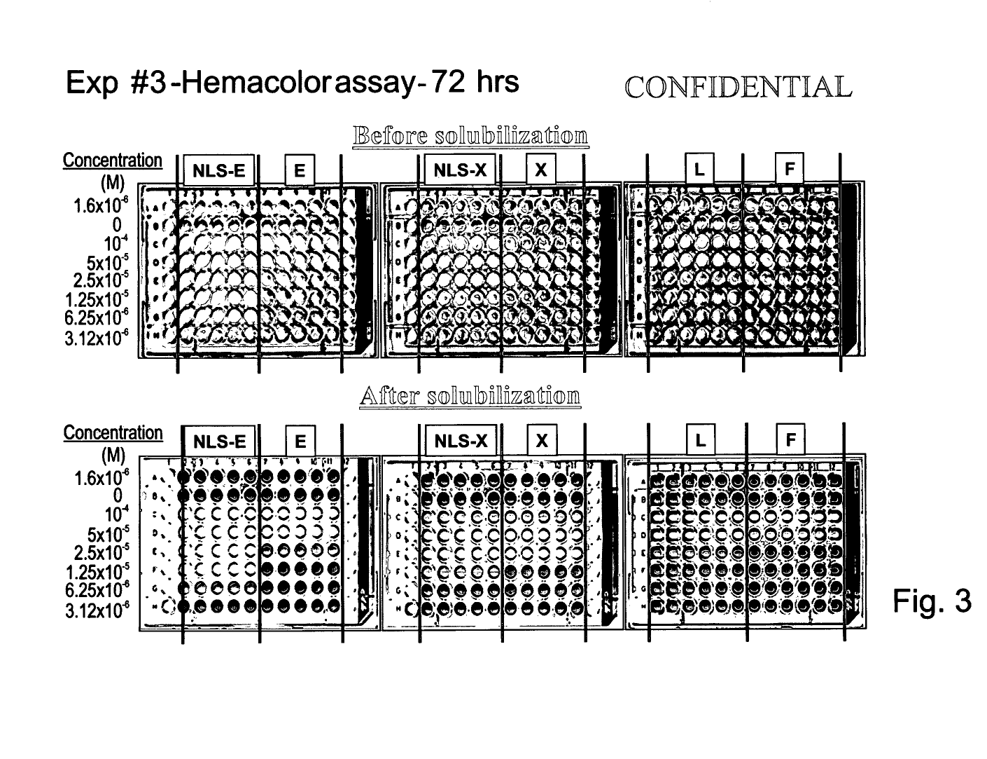

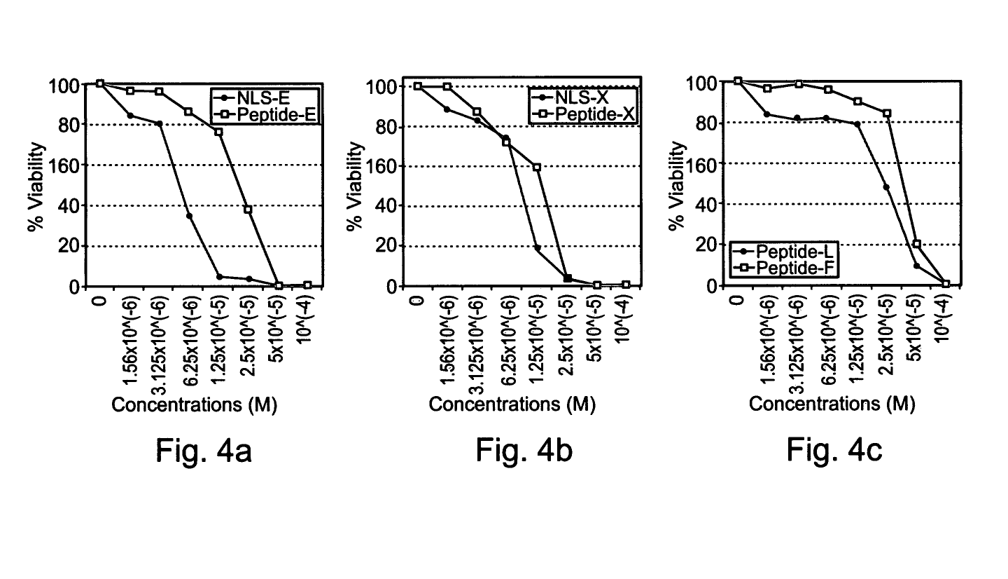

Table 10 below summarizes the concentration of the various peptides required to obtain 50% viability, or to destroy 50% of the tumor cells, as derived from the hemacolor test (FIGS. 3 and 4a-c). Of note, the higher the concentration of peptide required, the poorer the potency of the analog.

TABLE 10

| Peptide | Concentration [M] | Activity with respect to NLS-E (%) |
|---|---|---|
| NLS-E | 6 × 10⁻⁶ | 100 |
| NLS-X | 1 × 10⁻⁵ | 50 |
| X | 2 × 10⁻⁵ | 16 |
| E | 3.5 × 10⁻⁵ | 16 |
| L | 3.5 × 10⁻⁵ | 16 |
| F | 5 × 10⁻⁵ | 12.5 |

TABLE 11

| | Concentration [M] × 10⁻⁶ | Activity with respect to NLS |
|---|---|---|
| Peptide | (derived from table 10) | peptide (%) |
| NLS-E | 6 | |
| E | 35 | 16 |
| NLS-X | 10 | |
| X | 20 | 50 |

In view of the foregoing it is evident that the most potent peptide is NLS-E. Other peptides tested exhibited various growth inhibitory activities in the following order—NLS-E>NLS-X>X>E>L>F. As expected, the conjugation of the NLS improves peptides' activity.

Interestingly, the most potent peptides are palm-conjugated GnRH analogs which are clearly distinct from currently available non-conjugated GnRH analogs. They are characterized by high cytotoxic activity when applied to tumor cells which overexpress GnRH receptors (e.g. adenocarcinoma), without the need of using any cytotoxic moieties. As such, they can be used as valuable tools for the treatment GnRH-associated diseases in general and cancer in particular.

### Example 8

Selective Anti Adenocarcinoma Activity of the Peptides of the Present Invention

Selective destruction of adenocarcinoma cell line vs. normal cells was analysed on NLS-E.

Experimental Procedures

Hemacolor test on Colo-205 and on Human foreskin fibroblasts (ATCC CRL 2091) was effected as described in Example 5 above, in the presence of 1×10⁻⁵ M and 2.5×10⁻⁵M NLS-E peptide, where medium was used as control.

Results

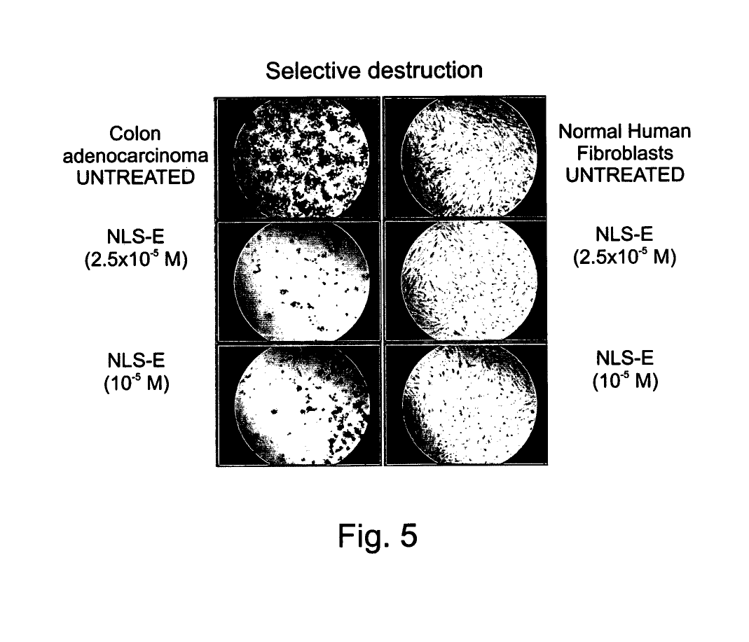

As is demonstrated in FIG. 5, cell destruction was observed already with 1×10⁻⁵ M NLS-E, and more dramatically which 2.5×10⁻⁵M NLS-E when incubated with the adenocarcinoma cell line. Normal human fibroblasts displayed resistance at both peptide concentrations.

### Example 9

### Screening for Anti-Cancer Peptide Activity on Various Adenocarcinoma Cell Lines

The peptides listed in Table 12 below were accepted by the Developmental Therapeutics Program of the National Cancer Institute (NCI) of the National Health Institutes (NIH) to be screened on 60 tumor cell lines.

TABLE 12

| Peptide | Structure | MW |
|---|---|---|
| 1. E | [(Palm-D-Lys-Pro-Gly)¹; D-Phe²; Pro³; D-Lys⁶]- | 1584.92 |
| | LH-RH; C₈₀H₁₃₀N₁₈O₁₅ | |
| 2. F | [(Palm-D-Lys-Gly)¹; D-Phe²; Pro³; D-Lys⁶]-LH- | 1486.92 |
| | RH; C₇₅H₁₂₃N₁₇O₁₄ | |
| 3. L | [(Palm-Pro-Gly)¹; D-Phe²; Ala³; D-Lys⁶]-LH-RH; | 1429.83 |
| | C₇₂H₁₁₆N₁₆O₁₄ | |
| 4. X | [(Palm-Pro-Gly)¹; D-Phe²; Pro³; D-Lys⁶]-LH-RH; | 1455.86 |
| | C₇₄H₁₁₈N₁₆O₁₄ | |
| 5. NLS-X | [(Pam-Pro-Gly)¹; D-Phe²; Pro³; D-Lys(H-Pro-Lys- | 2321.01 |
| | Lys-Lys-Arg-Lys-Val)⁶]-LH-RH; C₁₁₄H₁₉₄N₃₀O₂₁ | |
| 6. NLS-E | [(Palm-D-Lys(H-Pro-Lys-Lys-Lys-Arg-Lys- | 2449.18 |
| | Val)Pro-Gly)¹; D-Phe²; Pro³; D-Lys⁶]-LH-RH; | |
| | C₁₂₀H₂₀₆N₃₂O₂₂ | |

### Example 10

### Toxicology Analysis

### Example 10a

### Evaluation of the Total Tolerated Dosage (TTD) of Mice to a Single Injection of the Peptides X and NLS-E

### Test Compounds

The total tolerated dosage of peptide by mice administered with a single injection was tested.

Animals and Experimental Procedures

The test compounds were administered to 10 female athymic nude mice NCr-nu (Charles River Laboratories, Wilmington, Mass.) as a single injection at the following dosages and routes:

Peptide X was injected intra-peritonealy (IP) at 400, 200, and 100 mg/kg.

Peptide NLS-E was tested intra-venously (IV) at 400, 200, 100, 50 and 25 mg/kg

Different injection volumes of a stock solution at a concentration of 20 mg/ml were used to administer the desired dosages (injection volumes of 0.05, 0.1, and 0.2 ml/10 g body, respectively) The carrier was water for injection at pH 7. Mice were observed for 14 days following the injection for possible delayed toxicity. Individual body weights were measured twice a week.

Results

Peptide X injected IP was lethal at 400 and 200 mg/kg. Animals injected IP with 100 mg/kg stayed alive.

Peptide NLS-E injected IV was lethal at 400 200 and 100 mg/kg. Animals injected IV with 50 and 25 mg/kg stayed alive.

### Example 10b

### Evaluation of Maximal Tolerated Dosage (MTD) of Mice to the Peptides X and NLS-E

### Test Compounds

The maximum tolerated dosage (MTD) of the test compounds was tested when administered twice a day for 14 consecutive days.

Animals and Experimental Procedures

A total of 50 female athymic NCr-nu nude mice (obtained as mentioned in Example 10a) were used as follows: five groups of mice with five mice per group per compound. Peptides dissolved in water at pH 7 (the vehicle) were evaluated at four dosages (The dosages selected were derived from the results of the TTD experiment, Example 10a):

Peptide X was injected IP at 20 mg/kg/injection for 7 days, followed by IP injections at 60 mg/kg/injection 10, 5, 2.5 mg/kg/injection for 7 days followed by 30, 15 and 7.5 mg/kg, for 14 days respectively.

Peptide NLS-E was tested at 10, 5, 2.5, and 1.25 mg/kg/injection followed by injection at 30, 15, 7.5 and 3.75 mg/kg respectively. Injections were given IV except in the groups treated with 10/30 and 5/15 mg/kg/injection, where injections were switched to IP route on day 10 of treatment.

Additionally, one vehicle-treated control group of five mice per compound was included in the experiment. The approximate MTD was defined as the maximum dosage that does not produce lethality within 14 days of the last treatment or more than an average 20% weight loss. The MTD study was terminated on Day 29.

Mice were observed for 14 days after the injection for possible delayed toxicity. Individual body weights were measured twice a week.

Results

Three mice injected IP with 20/60 mg/kg of peptide X and one mouse injected with 10/30 mg/kg of peptide X, died. None of the IV injected NLS-E peptide died. Altogether, these results demonstrate that peptide NLS-E is less toxic than peptide X. For following in vivo assays with tumor bearing mice, peptide concentrations of 30, 15, and 7.5, and 3.75 mg/kg will be used. A vehicle-treated control group will be also included in the study.

### Example 11

### In-Vivo Anti-Tumor Activity of Peptides X and NLS-E

The anti-tumor activity of peptides X and NLS-E is evaluated in colo-205 bearing mice.

Animals and Experimental Procedures

Animals—Female athymic nude mice NCr-nu (Charles River Laboratories, Wilmington, Mass.) were implanted subcutaneously with 30-40 mg of fragments. Twenty days following fragment implantation, when the tumors reach 100-200 mg, 70 mice are treated as described in 10b.

Twenty days following fragment implantation, when the tumors reach 100-200 mg, 70 mice are treated as described in Example 10b.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

1-46. (canceled)

47. A peptide comprising at least one hydrophobic moiety attached to an amino acid sequence capable of binding a GnRH receptor, said amino acid sequence being at least 11 amino acids in length.

48. A peptide comprising at least one hydrophobic moiety attached to an amino acid sequence selected from the group consisting of SEQ ID NO: 2, 4, 5, 6, 7, 8, 9, 10, 12, 13, 14, 15, 16, 18, 21, 22, 23, 24, 27, 28, 32, 33, 34, 35 and 36.

49. A pharmaceutical composition comprising as an active ingredient the peptide of claim 47 and a pharmaceutical acceptable carrier or diluent.

50. A method of treating a GnRH associated disease in a subject the method comprising providing to a subject in need thereof a therapeutically effective amount of the peptide of claim 47, thereby treating the GnRH associated disease in the subject.

51. The peptide of claim 47, wherein said amino acid sequence is devoid of histidine and/or tryptophane.

52. The peptide of claim 47, wherein said hydrophobic moiety is a fatty acid.

53. The peptide of claim 47, wherein said hydrophobic moiety is selected from the group consisting of an aliphatic compound, alicyclic compound and an aromatic compound.

54. The peptide of claim 47, wherein said amino acid sequence includes the following general Formula:
X₁-X₂-X₃-Ser-Tyr-X₄-Leu-Arg-Pro

55. The peptide of claim 54, wherein X₄ is an amino group containing side chain amino acid.

56. The peptide of claim 54, wherein said amino acid sequence further includes an N-terminal amide group positioned C-terminally of said Pro.

57. The peptide of claim 54, wherein said X₁ is Gly or Pro.

58. The peptide of claim 54, wherein said X₂ is an aromatic amino acid.

59. The peptide of claim 54, wherein said X₃ is Pro, Ala or Trp.

60. The peptide of claim 54, wherein said amino acid sequence further includes an X₅ positioned N-terminally of X₁.

61. The peptide of claim 60, wherein said X₅ is selected from the group consisting of Pro and Lys.

62. The peptide of claim 54, wherein said amino acid sequence further includes an X₅ positioned between X₁ and X₂.

63. The peptide of claim 54, wherein said amino acid sequence further includes an X₅-X₆ dipeptide positioned N-terminally of X1.

64. The peptide of claim 47, wherein said peptide further includes a nuclear localization signal.

65. The peptide of claim 47, wherein said peptide further includes a cytotoxic agent attached thereto.

66. The peptide of claim 47, wherein said amino acid sequence is as set forth in SEQ ID NO: 2, 4, 5, 6, 7, 8, 9, 10, 12, 13, 14, 15, 16, 18, 21, 22, 23, 24, 27, 28, 32, 33, 34, 35 or 36.
